(19) United States
(12) Patent Application Publication
(10) Pub. No.: US 2024/0009315 A1
(43) Pub. Date: Jan. 11, 2024

(54) METHODS OF TARGETING AGENTS TO ACE2 RECEPTORS

(71) Applicant: Yeda Research and Development Co. Ltd., Rehovot (IL)

(72) Inventors: Gideon SCHREIBER, Rehovot (IL); Jiri ZAHRADNIK, Rehovot (IL); Amnon BAR-SHIR, Rehovot (IL); Yinon RUDICH, Rehovot (IL); Andrea GALISOVA, Rehovot (IL)

(73) Assignee: Yeda Research and Development Co. Ltd., Rehovot (IL)

(21) Appl. No.: 18/229,224

(22) Filed: Aug. 2, 2023

Related U.S. Application Data
(63) Continuation-in-part of application No. 18/122,169, filed on Mar. 16, 2023.
Application No. 18/122,169 is a continuation of application No. PCT/IL2021/051154, filed on Sep. 22, 2021.
(60) Provisional application No. 63/125,984, filed on Dec. 16, 2020.

(30) Foreign Application Priority Data
Sep. 23, 2020 (IL) 277546

(51) Int. Cl. A61K 47/64; A61K 47/69

(52) U.S. Cl. CPC A61K 47/64; A61K 47/6911

(57) ABSTRACT

Particles having an ACE2 targeting moiety attached to an outer surface thereof are disclosed herein. The ACE2 targeting moiety comprises a polypeptide comprising an amino acid sequence of SARS CoV-2 receptor-binding domain (RBD), wherein said amino acid sequence comprises a modification at position 358 and at least two additional modifications at two positions selected from the group consisting of 484, 498 and 501. Uses of the particles for treatment and diagnosis of diseases are also disclosed.

### RELATED APPLICATIONS

This application is a Continuation-In-Part of U.S. patent application Ser. No. 18/122,169 filed on Mar. 16, 2023, which is a Continuation of PCT Patent Application No. PCT/IL2021/051154 having International filing date of Sep. 22, 2021, which claims the benefit of priority of Israeli Patent Application No. 277546 filed on Sep. 23, 2020 and under 35 USC § 119(e) of U.S. Provisional Patent Application No. 63/125,984 filed on Dec. 16, 2020. The contents of the above applications are all incorporated by reference as if fully set forth herein in their entirety.

### SEQUENCE LISTING STATEMENT

The XML file, entitled 97395SequenceListing.xml, created on Aug. 2, 2023, comprising 66,544 bytes, submitted concurrently with the filing of this application is incorporated herein by reference.

### FIELD AND BACKGROUND OF THE INVENTION

The present invention, in some embodiments thereof, relates to method of targeting agents to angiotensin-converting enzyme 2 (ACE2) expressing cells and use thereof for treating or diagnosing disease.

Severe acute respiratory syndrome coronavirus 2 (SARS-CoV-2), the causative agent of coronavirus disease 2019 (COVID-19), targets lungs via its binding to angiotensin-converting enzyme 2 (ACE2) receptors, which are highly expressed at the surface of type 2 alveolar epithelial cells in the lungs. In addition, ACE2 is also expressed on other tissues, including the heart, kidney and small intestine. Virus entry into cells depends on the receptor-binding domain (RBD) of the SARS-CoV-2 spike protein (S) that specifically recognizes ACE2.

Extracellular vesicle (EVs) are natural transport nanovesicles secreted by numerous cell types. It is clear that EVs have the capacity to deliver specific cargo of functional biomolecules, such as nucleic acids (including plasmid DNA and siRNA), proteins and chemotherapeutic drugs, to the recipient cells, release cargo, and mediate many physiological or pathological processes. It is widely recognized that EVs are promising nanocarriers for clinical application based on their nanoscale for penetration into deep tissues, long-term circulation, and their naturally biocompatible characteristics. However, it has been shown that the majority of intravenous injected EVs are absorbed within the liver. Therefore, if EVs are candidates for the systemic delivery of therapeutic compounds and exploited as the targeted therapeutic particles, it will be necessary to deliver exogenous cargoes to specific tissues or cell types in vivo. Targeted EVs can be obtained by displaying targeting molecules, such as tissue-specific peptides or antibody fragments recognizing target antigens, on the outer surface of EVs.

Background art includes Fu et al., Journal of Controlled Release 335 (2021) 584-595 International Patent Application WO2022/064494, Galisova et al., ACS Nano 2022, 16, 12276-12289, Galisova et al., https://doi(dot)org/10.1101/2022.03.27.485958; Baig et al., Drugs in R&D (2020) 20:161-169; Starr et al., 2020, Cell 182, 1295-1310 and Huang et al., Acta Pharmacologica Sinica (2020) 41:1141-1149.

### SUMMARY OF THE INVENTION

According to an aspect of the present invention, there is provided a particle having an ACE2 targeting moiety attached to an outer surface thereof, wherein the ACE2 targeting moiety comprises a polypeptide comprising an amino acid sequence of SARS CoV-2 receptor-binding domain (RBD), wherein the amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein the polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein the polypeptide comprises at least 170 amino acids of the RBD.

According to embodiments of the invention, the ACE2 targeting moiety binds to ACE2 receptors on lung cells with at least 2 fold higher affinity than the wild-type RBD having an amino acid sequence as set forth in SEQ ID NO: 45, when assayed under identical conditions.

According to embodiments of the invention, the particle is attached to or encapsulates a therapeutic agent or a diagnostic agent.

According to embodiments of the invention, the therapeutic agent is selected from the group consisting of an antiviral agent, a cytotoxic agent, a bronchodilator, an antibiotic and an anti-inflammatory agent.

According to embodiments of the invention, the therapeutic agent or diagnostic agent is selected from the group consisting of a polynucleotide agent, a polypeptide agent and a small molecule agent.

According to embodiments of the invention, the particle is an extracellular vesicle (EV).

According to embodiments of the invention, the particle is a synthetic particle.

According to embodiments of the invention, the polypeptide comprises modifications at each of the positions 358, 484, 498 and 501, and optionally comprising a modification at position 460.

According to embodiments of the invention, the modification at position 358 comprises a I358F substitution, wherein the modification at position 484 comprises a E498K substitution, wherein the modification at position 498 comprises a Q498R substitution, or the modification at position 501 comprises a N501Y substitution.

According to embodiments of the invention, the polypeptide comprises the substitutions:
(i) I358F, N460K, E484K, S494P, Q498R and N501Y;
(ii) I358F, N460K, E484K, Q498R and N501Y;
(iii) I358F, E484K, Q498R and N501Y;
(iv) I358F, V445K, N460K, I468T, T470M, S477N, E484K, Q498R and N501Y; or
(v) I358F, V367W, R408D, K417V, V445K, N460K, I468T, T470M, S477N, E484K, Q498R and N501Y.

According to embodiments of the invention, the polypeptide comprises an amino acid sequence at least 99% identical to SEQ ID NO: 38, 39 or 40.

According to embodiments of the invention, the polypeptide comprises no more than 250 amino acids of the S1 subunit of the spike protein of SARS CoV-2.

According to embodiments of the invention, the polypeptide is a dimer

According to an aspect of the present invention, there is provided a method of treating an ACE2-associated disease in a subject in need thereof, comprising administering to the subject a therapeutically effective amount of a particle having an ACE2 targeting moiety attached to an outer surface thereof, wherein the ACE2 targeting moiety comprises a polypeptide comprising an amino acid sequence of SARS CoV-2 receptor-binding domain (RBD), wherein the amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein the polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein the polypeptide comprises at least 170 amino acids of the RBD, wherein the particle is attached to, or encapsulates, a therapeutic agent, thereby treating the disease.

According to an aspect of the present invention, there is provided a method of diagnosing an ACE2-associated disease in a subject in need thereof, comprising administering to the subject an effective amount of a particle having an ACE2 targeting moiety attached to an outer surface thereof, wherein the ACE2 targeting moiety comprises a polypeptide comprising an amino acid sequence of SARS CoV-2 receptor-binding domain (RBD), wherein the amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein the polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein the polypeptide comprises at least 170 amino acids of the RBD, wherein the particle is attached to, or encapsulates, a diagnostic agent, thereby diagnosing the disease.

According to embodiments of the invention, the disease is a respiratory disease.

According to embodiments of the invention, the administering is effected by inhalation.

According to embodiments of the invention, the respiratory disease is a respiratory infection.

According to embodiments of the invention, the therapeutic agent is selected from the group consisting of a polynucleotide agent, a polypeptide agent and a small molecule agent.

According to embodiments of the invention, the therapeutic agent is selected from the group consisting of an antiviral agent, an antibiotic, a cytotoxic agent, a bronchodilator and an anti-inflammatory agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A. Schematic illustration of the experiment depicting the binding of EVs^{RBD} to ACE2 expressing at the surface of cells. Location of the RBD domain mutations characteristic of the Wuhan and predicted “62” mutation of SARS-CoV-2 RBD as depicted in the Wuhan structure (pdb 6m17:f); the variations from the Wuhan variant are shown as blue circles.

FIG. 1B. Flow cytometry analysis of ACE2-expressing cells (n=3) incubated with fluorescently labeled EVs: EVs^{noRBD}, EVs^{RBD}, and EVs^{RBD-62}. Data are presented as mean values ±s.d. Statistics: two-tailed unpaired Student's t-test with *p-value<0.05, **p-value<0.01, *** p<0.001, and ****p<0.0001.

### DESCRIPTION OF SPECIFIC EMBODIMENTS OF THE INVENTION

The present invention, in some embodiments thereof, relates to method of targeting agents to angiotensin-converting enzyme 2 (ACE2) expressing cells and use thereof for treating or diagnosing disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The mechanism of entry of SARS-CoV-2 is through binding the ACE2 protein that is anchored on the membrane of cells of the respiratory system. The present inventors previously engineered ultra-tight ACE2 binding proteins, with the binding being almost non-reversible. The binding proteins, which are based on the natural receptor-binding domain (RBD) of the spike protein (S-protein) of SARS-CoV-2, show >1000-fold tighter binding than the wild-type (WT) RBD).

The present inventors now propose that these binding proteins can be used for targeting agents to cells expressing the ACE2 protein. Since ACE2 is found in cells of numerous tissues including lung, heart, kidney and small intestine, the proteins can be used for therapeutic treatment and diagnosis of a wide variety of diseases.

Thus, according to a first aspect of the present invention, there is provided a particle having an ACE2 targeting moiety attached to an outer surface thereof, wherein said ACE2 targeting moiety comprises a polypeptide comprising an amino acid sequence of SARS CoV-2 receptor-binding domain (RBD), wherein said amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein said polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein said polypeptide comprises at least 170 amino acids of said RBD.

As used herein “SARS-CoV-2 receptor binding domain (RBD)” refers to the receptor (ACE2) binding domain of SARS-CoV-2 of SPIKE, residues Arg319-Phe541 of SPIKE (as set forth in SEQ ID NO: 45). The full length amino acid sequence of SPIKE is set forth in SEQ ID NO: 37.

The polypeptides described herein bind to wild-type ACE2 receptors which are naturally found on the cell surface (e.g. on lung cells). In one embodiment, the polypeptides described herein bind to wild-type ACE2 receptors which are naturally found on the cell surface (e.g. on lung cells) with at least 2, 5 fold, 10 fold, 20 fold, 50 fold higher affinity than the wild-type RBD having an amino acid sequence as set forth in SEQ ID NO: 45, when assayed under identical conditions (either in-vitro and/or in vivo).

As used herein “ACE2” refers to Angiotensin-converting enzyme 2 (ACE2) E.C. 3.4.17.23 (GenBank Accession No. NP_068576), which is encoded in human by the ACE2 gene. ACE2 is an enzyme attached to the cell membranes of cells in the lungs, arteries, heart, kidney, and intestines. ACE2 lowers blood pressure by catalysing the hydrolysis of angiotensin II (a vasoconstrictor peptide) into angiotensin (1-7) (a vasodilator). ACE2 counters the activity of the related angiotensin-converting enzyme (ACE) by reducing the amount of angiotensin-II and increasing Ang(1-7). ACE2 also serves as the entry point into cells for Coronaviruses, including HCoV-NL63, SARS-CoV, and SARS-CoV-2. The human version of the enzyme is often referred to as hACE2.

According to a specific embodiment, the ACE2 is human ACE2.

In order to express the polypeptides of this aspect of the present invention on the surface of yeast cells (e.g. Pichia pastoris or Saccharomyces cerevisiae cells), preferably they are expressed as fusion proteins together with anchoring protein such as yeast mating agglutinin factor A protein, as known in the art. Other yeast anchoring proteins include, but are not limited to Cwp1, Cwp2, Gas1p, Yap3p, Flo1p, Crh2p, Pir1, Pir2, Pir4, Icwp, HpSEDI, HpGASI, HpTIPI, HPWPI, HwpIp, Als3p, Rbt5p.

Vectors for expressing the polypeptides in yeast cells include Ylp-based vectors, such as Ylp5, YRp vectors, such as YRp17, YBp vectors such as YEp13 and YCp vectors, such as YCp19. Other examples of the YEp vectors include YEp24, YEp51, and YBp52, which are cloning and expression vehicles useful in the introduction of genetic constructs into S. cerevisiae (see, e.g., Broach et al. (1983) in Experimental Manipulation of Gene Expression, ed. M. Inouye Academic Press, p. 83). These vectors are also shuttle vectors in that they can replicate in E. coli due the presence of the pBR322 ori, and in S. cerevisiae due to the replication determinant of the yeast 2 micron plasmid.

Suitable promoters for function in yeast include the promoters for metallothionein, 3-phosphoglycerate kinase (Hitzeman et al., J. Biol. Chem. 255, 2073 (1980) or other glycolytic enzymes (Hess et al., J. Adv. Enzyme Req. 7, 149 (1968); and Holland et al. Biochemistry 17, 4900 (1978)), such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phospho-glucose isomerase, and glucokinase. Suitable vectors and promoters for use in yeast expression are further described in R. Hitzeman et al., EP073, 657. Other suitable promoters for expression in yeast include the promoters from GAL1 (galactose), PGK (phosphoglycerate kinase), ADH (alcohol dehydrogenase), AOX1 (alcohol oxidase), HIS4 (histidinol dehydrogenase), and the like. Still other promoters, which have the additional advantage of transcription controlled by growth conditions, are the promoter regions for alcohol dehydrogenase 2, isocytochrome C, acid phosphatase, degradative enzymes associated with nitrogen metabolism, and the afore-mentioned metallothionein and glyceraldehyde-3-phosphate dehydrogenase, as well as enzymes responsible for maltose and galactose utilization. Finally, promoters that are active in only one of the two haploid mating types may be appropriate in certain circumstances. Among these hapioid-specific promoters, the pheromone promoters MFa1 and MFα1 are of particular interest.

Once the amount of polypeptide displayed on the yeast surface is determined (e.g. using a fluorescent protein which is co-expressed with the polypeptide, as described in the Examples section herein below, or via a labelled antibody, as known in the art), it is possible to determine the binding affinity for soluble ACE2. Analysis of fluorescence may be carried out as known in the art (for example by Fluorescence Activated Cell Sorting (FACS or flow cytometry).

As mentioned, the polypeptides of this aspect of the present invention, bind soluble, monomeric angiotensin-converting enzyme 2 (ACE2) receptor, when expressed on the surface of yeast cells, with at least 50 fold, 100 fold, 200 fold, 300 fold, 400 fold, 500 fold, 600 fold, 700 fold, 800 fold, 900 fold or even 1000 fold higher affinity than the wild-type RBD having an amino acid sequence as set forth in SEQ ID NO: 37, when assayed under identical conditions.

As used herein “soluble” refers to the portion of ACE2 which is devoid of a transmembrane domain (as defined by coordinates 741-761 of the human ACE2 protein) and the membrane proximal extracellular portion 616-741, and optionally also the intracellular domain (762 till end 805). Amino acid coordinates correspond to SEQ ID NO: 44.

In particular, the polypeptides of this aspect of the present invention bind to ACE2 with a K_{D} of less than 200 μM, less than 20 μM and even less than 10 μM, as measured in a yeast display assay.

As used herein the term “K_{D}” refers to the equilibrium dissociation constant between the polypeptide variant of the RBD of Spike and hACE2.

According to a specific embodiment, the K_{D} is below 0.3 nM (e.g., 0.01-0.1 nM, 0.01-0.09 nM, 0.01-0.08 nM, 0.01-0.07 nM, 0.01-0.06 nM, 0.01-0.05 nM, 0.01-0.04 nM, 0.01-0.03 nM, 0.01-0.02 nM, 0.01 nM), as determined by ForteBio's Octet® RED96 Surface interferometry e.g., where hACE2 is the SOLUBLE analyte.

According to a particular embodiment, the RBD polypeptides of this aspect of the present invention comprises at least 170 consecutive amino acids of the native sequence of the RBD of SARS-CoV-2, at least 180 consecutive amino acids of the native sequence of RBD of SARS-CoV-2, at least 190 consecutive amino acids of the native sequence of RBD of SARS-CoV-2, at least 200 consecutive amino acids of the native sequence of RBD of SARS-CoV-2, at least 210 consecutive amino acids of the native sequence of RBD of SARS-CoV-2, at least 220 consecutive amino acids of the native sequence of RBD of SARS-CoV-2. For example, the RBD polypeptides described herein preferably comprise amino acids 330-516 of the RBD of the S1 subunit of the spike protein of SARS CoV-2.

For the purpose of this invention, the term “consecutive amino acids” also includes the mutations which are disclosed herein.

According to additional embodiments, the polypeptides comprise no more than 250 amino acids of the S1 subunit of the spike protein of SARS CoV-2.

The polypeptides described herein are typically no longer than 250 amino acids, 260 amino acids, 270 amino acids, 280 amino acids, 290 amino acids or 300 amino acids.

The terms “peptide” and “polypeptide” which are interchangeably used herein encompass native peptides backbone (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body, more capable of penetrating into cells improving clearance, biodistribution and/or pharmacokinetics. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (e.g. 2-3) bonds at the same time.

Natural aromatic amino acids, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables 1 and 2 below list naturally occurring amino acids (Table 1), and non-conventional or modified amino acids (e.g., synthetic, Table 2) which can be used with some embodiments of the invention.

TABLE 1

| Amino Acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code |
|---|---|
| ornithine | Orn |
| α-aminobutyric acid | Abu |
| D-alanine | Dala |
| D-arginine | Darg |
| D-asparagine | Dasn |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-N-methylalanine | Dnmala |
| D-N-methylarginine | Dnmarg |
| D-N-methylasparagine | Dnmasn |
| D-N-methylasparatate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| D-N-methylmethionine | Dnmmet |
| D-N-methylornithine | Dnmorn |
| D-N-methylphenylalanine | Dnmphe |
| D-N-methylproline | Dnmpro |
| D-N-methylserine | Dnmser |
| D-N-methylthreonine | Dnmthr |
| D-N-methyltryptophan | Dnmtrp |
| D-N-methyltyrosine | Dnmtyr |
| D-N-methylvaline | Dnmval |
| L-norleucine | Nle |
| L-norvaline | Nva |
| L-ethylglycine | Etg |
| L-t-butylglycine | Tbug |
| L-homophenylalanine | Hphe |
| α-naphthylalanine | Anap |
| penicillamine | Pen |
| Y-aminobutyric acid | Gabu |
| cyclohexylalanine | Chexa |
| cyclopentylalanine | Cpen |
| α-amino-α-methylbutyrate | Aabu |
| γ-aminoisobutyric acid | Aib |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methyl glutamic acid | Dmglu |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmtyr |
| D-α-methylvaline | Dmval |
| N-cyclobutylglycine | Ncbut |
| N-cycloheptylglycine | Nchep |
| N-cyclohexylglycine | Nchex |
| N-cyclodecylglycine | Ncdec |
| N-cyclododecylglycine | Ncdod |
| N-cyclooctylglycine | Ncoct |
| N-cyclopropylglycine | Ncpro |
| N-cycloundecylglycine | Ncund |
| N-(2-aminoethyl)glycine | Naeg |
| N-(2,2-diphenylethyl)glycine | Nbhm |
| N-(3,3-diphenylpropyl)glycine | Nbhe |
| 1-carboxy-1-(2,2-diphenyl | Nmbc |
| ethylamino)cyclopropane | |
| phosphoserine | pSer |
| phosphotyrosine | pTyr |
| 2-aminoadipic acid | |
| hydroxyproline | Hyp |
| aminonorbornyl- | Norb |
| carboxylate | |
| aminocyclopropane- | Cpro |
| carboxylate | |
| N-(3-guanidinopropyl)glycine | Narg |
| N-(carbamylmethyl)glycine | Nasn |
| N-(carboxymethyl)glycine | Nasp |
| N-(thiomethyl)glycine | Ncys |
| N-(2-carbamylethyl)glycine | Ngln |
| N-(2-carboxyethyl)glycine | Nglu |
| N-(imidazolylethyl)glycine | Nhis |
| N-(1-methylpropyl)glycine | Nile |
| N-(2-methylpropyl)glycine | Nleu |
| N-(4-aminobutyl)glycine | Nlys |
| N-(2-methylthioethyl)glycine | Nmet |
| N-(3-aminopropyl)glycine | Norn |
| N-benzylglycine | Nphe |
| N-(hydroxymethyl)glycine | Nser |
| N-(1-hydroxyethyl)glycine | Nthr |
| N-(3-indolylethyl) glycine | Nhtrp |
| N-(p-hydroxyphenyl)glycine | Ntyr |
| N-(1-methylethyl)glycine | Nval |
| N-methylglycine | Nmgly |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylnorleucine | Nmnle |
| L-N-methylnorvaline | Nmnva |
| L-N-methyl-ethylglycine | Nmetg |
| L-N-methyl-t-butylglycine | Nmtbug |
| L-N-methyl-homophenylalanine | Nmhphe |
| N-methyl-α-naphthylalanine | Nmanap |
| N-methylpenicillamine | Nmpen |
| N-methyl-γ-aminobutyrate | Nmgabu |
| N-methyl-cyclohexylalanine | Nmchexa |
| N-methyl-cyclopentylalanine | Nmcpen |
| N-methyl-α-amino-α- | Nmaabu |
| methylbutyrate | |
| N-methyl-α-aminoisobutyrate | Nmaib |
| L-α-methylarginine | Marg |
| L-α-methylasparagine | Masn |
| L-α-methylaspartate | Masp |
| L-α-methylcysteine | Mcys |
| L-α-methylglutamine | Mgln |
| L-α-methylglutamate | Mglu |
| L-α-methylhistidine | Mhis |
| L-α-methylisoleucine | Mile |
| L-α-methylleucine | Mleu |
| L-α-methyllysine | Mlys |
| L-α-methylmethionine | Mmet |
| L-α-methylornithine | Morn |
| L-α-methylphenylalanine | Mphe |
| L-α-methylproline | Mpro |
| L-α-methylserine | Mser |
| L-α-methylthreonine | Mthr |
| L-α-methyltryptophan | Mtrp |
| L-α-methyltyrosine | Mtyr |
| L-α-methylvaline | Mval |
| L-α-methylnorvaline | Mnva |
| L-α-methylethylglycine | Metg |
| L-α-methyl-t-butylglycine | Mtbug |
| L-α-methyl-homophenylalanine | Mhphe |
| α-methyl-α-naphthylalanine | Manap |
| α-methylpenicillamine | Mpen |
| α-methyl-γ-aminobutyrate | Mgabu |
| α-methyl-cyclohexylalanine | Mchexa |
| α-methyl-cyclopentylalanine | Mcpen |
| N-(N-(2,2-diphenylethyl) | Nnbhm |
| carbamylmethyl-glycine | |
| N-(N-(3,3-diphenylpropyl) | Nnbhe |
| carbamylmethyl-glycine | |
| 1,2,3,4-tetrahydroisoquinoline-3- | Tic |
| carboxylic acid | |
| phosphothreonine | pThr |
| O-methyl-tyrosine | |
| hydroxylysine | |

The amino acids of the polypeptides of some embodiments of the present invention may be substituted either conservatively or non-conservatively.

The term “conservative substitution” as used herein, refers to the replacement of an amino acid present in the native sequence in the polypeptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOH]—CO— for aspartic acid. Those non-conservative substitutions which fall under the scope of the present invention are those which still constitute a polypeptide capable of binding SPIKE.

The polypeptides of some embodiments of the invention are preferably utilized in a linear form, although it will be appreciated that in cases where cyclicization does not severely interfere with polypeptide characteristics, cyclic forms of the polypeptide can also be utilized.

The peptides of some embodiments of the invention preferably include one or more non-natural or natural polar amino acids, including but not limited to serine and threonine which are capable of increasing polypeptide solubility due to their hydroxyl-containing side chain.

Following is a description of amino acid mutations, which may be preferably employed.

As used herein, the phrase “plurality of mutations” refer to 3-20, 3-15, 3-10, 3-8, 3-6, 3-5, 4 or 3 mutations (with respect to the human sequence) which affect the KD of the polypeptide to soluble monomeric ACE2.

According to a specific embodiment, the plurality of mutations refers to 3, 4, 5, 6, 7, 8, 9, 10, 11, 12, 13, 14, 15, 16, 17, 18, 19 or 20 mutations.

As used herein “mutation” refers to any mutation (e.g., amino acid substitution, deletion, insertion). According to a specific embodiment, the mutation is a point mutation. According to a specific embodiment, the mutation is a substitution. The definition of mutation is with reference to SEQ ID NO: 37.

In one embodiment, the modification is at position 358 and at least two additional positions 484, 498 and 501.

In another embodiment, the modification is at each of the positions 358, 484, 498 and 501.

In one alternative, the modification further comprises one at position 460.

An exemplary modification at position 358 comprises a I358F substitution, an exemplary modification at position 460 comprises a N460K substitution, an exemplary modification at position 484 comprises a E498K substitution, an exemplary modification at position 498 comprises a Q498R substitution, and an exemplary modification at position 501 comprises a N501Y substitution.

Modifications (substitutions) for exemplary polypeptides are detailed herein below:
(i) I358F, N460K, E484K, S494P, Q498R and N501Y;
(ii) I358F, N460K, E484K, Q498R and N501Y;
(iii) I358F, E484K, Q498R and N501Y;
(iv) I358F, V445K, N460K, I468T, T470M, S477N, E484K, Q498R and N501Y; or
(v) I358F, V367W, R408D, K417V, V445K, N460K, I468T, T470M, S477N, E484K, Q498R and N501Y.

Thus, according to a specific embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 38 as determined using the Standard protein-protein BLAST [blastp] software of the NCBI, with the restriction that the amino acid at position 358 is F and not replaceable, the amino acid at position 460 is K and not replaceable, the amino acid at position 484 is K and not replaceable, the amino acid at position 494 is P and not replaceable, that the amino acid at position 498 is R and not replaceable, the amino acid at position 501 is Y and not replaceable. This polypeptide is referred to herein as B52.

In another embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 39 as determined using the Standard protein-protein BLAST [blastp] software of the NCBI, with the restriction that the amino acid at position 358 is F and not replaceable, the amino acid at position 445 is K and not replaceable, the amino acid at position 460 is K and not replaceable, the amino acid at position 468 is T and not replaceable, the amino acid at position 470 is M and not replaceable, the amino acid at position 477 is N and not replaceable, the amino acid at position 484 is K and not replaceable, the amino acid at position 498 is R and not replaceable, the amino acid at position 501 is Y and not replaceable. This polypeptide is referred to herein as B62.

In another embodiment, the polypeptide comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 40 as determined using the Standard protein-protein BLAST [blastp] software of the NCBI, with the restriction that the amino acid at position 358 is F and not replaceable, the amino acid at position 367 is W and not replaceable, the amino acid at position 408 is D and not replaceable, the amino acid at position 417 is V and not replaceable, the amino acid at position 445 is K and not replaceable, the amino acid at position 460 is K and not replaceable, the amino acid at position 468 is T and not replaceable, the amino acid at position 470 is M and not replaceable, the amino acid at position 477 is N and not replaceable, the amino acid at position 484 is K and not replaceable, the amino acid at position 498 is R and not replaceable, the amino acid at position 501 is Y and not replaceable. This polypeptide is referred to herein as B71.

According to another embodiment, the polypeptide comprises a protecting moiety or a stabilizing moiety.

The term “protecting moiety” refers to any moiety (e.g. chemical moiety) capable of protecting the polypeptide from adverse effects such as proteolysis, degradation or clearance, or alleviating such adverse effects.

The term “stabilizing moiety” refers to any moiety (e.g. chemical moiety) that inhibits or prevents a polypeptide from degradation.

The addition of a protecting moiety or a stabilizing moiety to the polypeptide typically results in masking the charge of the polypeptide terminus, and/or altering chemical features thereof, such as, hydrophobicity, hydrophilicty, reactivity, solubility and the like. Examples of suitable protecting moieties can be found, for example, in Green et al., “Protective Groups in Organic Chemistry”, (Wiley, 2.sup.nd ed. 1991) and Harrison et al., “Compendium of Synthetic Organic Methods”, Vols. 1-8 (John Wiley and Sons, 1971-1996).

The protecting moiety (or group) or stabilizing moiety (or group) may be added to the N-(amine) terminus and/or the C- (carboxyl) terminus of the polypeptide.

Representative examples of N-terminus protecting/stabilizing moieties include, but are not limited to, formyl, acetyl (also denoted herein as “Ac”), trifluoroacetyl, benzyl, benzyloxycarbonyl (also denoted herein as “CBZ”), tert-butoxycarbonyl (also denoted herein as “BOC”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “FMOC”), nitro-veratryloxycarbonyl (also denoted herein as “NVOC”), t-amyloxycarbonyl, adamantyl-oxycarbonyl, and p-methoxybenzyloxycarbonyl, 2-chlorobenzyloxycarbonyl and the like, nitro, tosyl (CH3C6H4SO2-), adamantyloxycarbonyl, 2,2,5,7, 8-pentamethylchroman-6-sulfonyl, 2,3,6-trimethyl-4-methoxyphenylsulfonyl, t-butyl benzyl (also denoted herein as “BZL”) or substituted BZL, such as, p-methoxybenzyl, p-nitrobenzyl, p-chlorobenzyl, o-chlorobenzyl, 2,6-dichlorobenzyl, t-butyl, cyclohexyl, cyclopentyl, benzyloxymethyl (also denoted herein as “BOM”), tetrahydropyranyl, chlorobenzyl, 4-bromobenzyl, and 2,6-dichlorobenzyl.

According to one embodiment of the invention, the protecting/stabilizing moiety is an amine protecting moiety.

According to a specific embodiment, the protecting/stabilizing moiety is a terminal cysteine residue.

Representative examples of C-terminus protecting/stabilizing moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively the —COOH group of the C-terminus may be modified to an amide group.

Other modifications of polypeptides include replacement of the amine and/or carboxyl with a different moiety, such as hydroxyl, thiol, halide, alkyl, aryl, alkoxy, aryloxy and the like.

According to a specific embodiment, the protecting/stabilizing moiety is an amide.

According to a specific embodiment, the protecting/stabilizing moiety is a terminal cysteine residue.

According to one embodiment, the protecting/stabilizing moiety comprises at least one, two, three or more cysteine residues at the N- or C-termini of the polypeptide.

Also included in the scope of the present invention are “chemical derivative” of a polypeptide or analog. Such chemical derivates contain additional chemical moieties not normally a part of the polypeptide. Covalent modifications of the polypeptide are included within the scope of this invention. Such modifications may be introduced into the molecule by reacting targeted amino acid residues of the polypeptide with an organic derivatizing agent that is capable of reacting with selected side chains or terminal residues. Many such chemical derivatives and methods for making them are well known in the art, some are discussed hereinbelow.

Also included in the scope of the invention are salts of the polypeptides and analogs of the invention. As used herein, the term “salts” refers to both salts of carboxyl groups and to acid addition salts of amino groups of the polypeptide molecule. Salts of a carboxyl group may be formed by means known in the art and include inorganic salts, for example, sodium, calcium, ammonium, ferric or zinc salts, and the like, and salts with organic bases such as those formed for example, with amines, such as triethanolamine, arginine, or lysine, piperidine, procaine, and the like. Acid addition salts include, for example, salts with mineral acids such as, for example, hydrochloric acid or sulfuric acid, and salts with organic acids, such as, for example, acetic acid or oxalic acid. Such chemical derivatives and salts are preferably used to modify the pharmaceutical properties of the polypeptide insofar as stability, solubility, etc., are concerned.

The polypeptides of this aspect of present invention can be biochemically synthesized such as by using standard solid phase techniques. These methods include exclusive solid phase synthesis, partial solid phase synthesis methods, fragment condensation and classical solution synthesis. These methods are preferably used when the polypeptide is relatively short (i.e., 10 kDa) and/or when it cannot be produced by recombinant techniques (i.e., not encoded by a nucleic acid sequence) and therefore involve different chemistry.

A particular method of preparing the polypeptide compounds of some embodiments of the invention involves solid phase polypeptide synthesis.

Large scale polypeptide synthesis is described by Andersson Biopolymers 2000; 55(3):227-50.

Synthetic polypeptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the polypeptides of the present invention are desired, the polypeptides of the present invention can be generated using recombinant techniques such as described by Bitter et al. (1987) Methods in Enzymol. 153:516-544; Studier et al. (1990) Methods in Enzymol. 185:60-89; Brisson et al. (1984) Nature 310:511-514; Takamatsu et al. (1987) EMBO J. 6:307-311; Coruzzi et al. (1984) EMBO J. 3:1671-1680; Brogli et al. (1984) Science 224:838-843; Gurley et al. (1986) Mol. Cell. Biol. 6:559-565 and Weissbach & Weissbach, 1988, Methods for Plant Molecular Biology, Academic Press, NY, Section VIII, pp 421-463.

For example, a nucleic acid sequence encoding an isolated polypeptide of the present invention (e.g., the amino acid sequences set forth in SEQ ID NOs: 38, 39 or 40) is ligated to a nucleic acid sequence which may include an inframe sequence encoding a proteinaceous moiety such as immunoglobulin.

Exemplary nucleic acid sequences which may be used to express the polypeptides in yeast cells are set forth in SEQ ID NOs: 46, 47 or 48.

Also provided is an expression vector, comprising the isolated polynucleotide of some embodiments of the invention. According to one embodiment, the polynucleotide sequence is operably linked to a cis-acting regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion from a host cell in which it is placed. Preferably the signal sequence for this purpose is a mammalian signal sequence.

Preferably, the promoter utilized by the nucleic acid construct of some embodiments of the invention is active in the specific cell population transformed.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Also provided are cells which comprise the polynucleotides/expression vectors as described herein.

Suitable host cells for cloning or expression include prokaryotic or eukaryotic cells. See e.g. Charlton, Methods in Molecular Biology, Vol. 248 (B. K. C. Lo, ed., Humana Press, Totowa, N. J., 2003), pp. 245-254, describing expression of antibody fragments in E. coli; see Gerngross, Nat. Biotech. 22:1409-1414 (2004), and Li et al., Nat. Biotech. 24:210-215 (2006) for suitable fungi and yeast strains; and see e.g., U.S. Pat. Nos. 5,959,177, 6,040,498, 6,420,548, 7,125,978, and 6,417,429 for suitable plant cell cultures which can also be utilized as hosts.

After expression, the isolated polypeptide may be isolated from the cells in a soluble fraction and can be further purified.

Recovery of the isolated polypeptide may be effected following an appropriate time in culture. The phrase “recovering the recombinant polypeptide or fusion protein” refers to collecting the whole fermentation medium containing the polypeptide or fusion protein and need not imply additional steps of separation or purification.

Notwithstanding the above, proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Proteins of the present invention are preferably retrieved in “substantially pure” form. As used herein, “substantially pure” refers to a purity that allows for the effective use of the protein in the applications, described herein.

Once polypeptides are obtained, they may be tested for binding affinity as discussed in detail above.

In one embodiment, the ACE2 targeting moiety is a dimer comprising two monomers linked by a linker, wherein each of said two monomers comprises an amino acid sequence encoding SARS CoV-2 receptor-binding domain (RBD), wherein each of said monomers binds soluble, monomeric angiotensin-converting enzyme 2 receptor when expressed on the surface of yeast cells with at least 50 fold higher affinity than the wild-type RBD having an amino acid sequence as set forth in SEQ ID NO: 45, when assayed under identical conditions.

In some embodiments, the dimer is such that the amino acid sequence of each of its monomers are the same, thus forming a homodimeric peptide.

In some embodiments, the dimer is such that the amino acid sequence of each of its peptide monomers are different, thus forming a heterodimeric peptide.

As mentioned, the monomers of the dimer of this aspect of the present invention are derived from the SARS CoV-2 receptor binding domain. In a particular embodiment, the monomers comprise the mutations described herein above (for example the replacement at position 358—I358F).

According to a specific embodiment, the first and second monomer of the dimer comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 38 as determined using the Standard protein-protein BLAST [blastp] software of the NCBI, with the restriction that the amino acid at position 358 is F and not replaceable, the amino acid at position 460 is K and not replaceable, the amino acid at position 484 is K and not replaceable, the amino acid at position 494 is P and not replaceable, that the amino acid at position 498 is R and not replaceable, the amino acid at position 501 is Y and not replaceable. This polypeptide is referred to herein as B52.

In another embodiment, the first and second monomer of the dimer comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 39 as determined using the Standard protein-protein BLAST [blastp] software of the NCBI, with the restriction that the amino acid at position 358 is F and not replaceable, the amino acid at position 445 is K and not replaceable, the amino acid at position 460 is K and not replaceable, the amino acid at position 468 is T and not replaceable, the amino acid at position 470 is M and not replaceable, the amino acid at position 477 is N and not replaceable, the amino acid at position 484 is K and not replaceable, the amino acid at position 498 is R and not replaceable, the amino acid at position 501 is Y and not replaceable. This polypeptide is referred to herein as B62.

In another embodiment, the first and second monomer of the dimer comprises a sequence as least 90% homologous, at least 91% homologous/identical, at least 92% homologous/identical, at least 93% homologous/identical, at least 94% homologous/identical, at least 95% homologous/identical, at least 96% homologous/identical, at least 97% homologous/identical, at least 98% homologous/identical, at least 99% homologous/identical, 100% homologous/identical to the sequence as set forth in SEQ ID NO: 40 as determined using the Standard protein-protein BLAST [blastp] software of the NCBI, with the restriction that the amino acid at position 358 is F and not replaceable, the amino acid at position 367 is W and not replaceable, the amino acid at position 408 is D and not replaceable, the amino acid at position 417 is V and not replaceable, the amino acid at position 445 is K and not replaceable, the amino acid at position 460 is K and not replaceable, the amino acid at position 468 is T and not replaceable, the amino acid at position 470 is M and not replaceable, the amino acid at position 477 is N and not replaceable, the amino acid at position 484 is K and not replaceable, the amino acid at position 498 is R and not replaceable, the amino acid at position 501 is Y and not replaceable. This polypeptide is referred to herein as B71.

According to another embodiment, the first monomer of the dimer is B52 (or homologs thereof, as defined herein above) and the second is B62 (or homologs thereof, as defined herein above).

According to another embodiment, the first monomer of the dimer is B52 (or homologs thereof, as defined herein above) and the second is B71 (or homologs thereof, as defined herein above).

According to another embodiment, the first monomer of the dimer is B62 (or homologs thereof, as defined herein above) and the second is B71 (or homologs thereof, as defined herein above).

According to a specific embodiment, the dimer comprises an amino acid sequence at least 95% identical/homologous to SEQ ID NO: 43.

Linking of the monomers of the peptide may be effected using any method known in the art provided that the linking does not substantially interfere with the bioactivity of the multimeric peptide—e.g. to interfere with the ability of the dimer to bind to ACE2.

The monomers of this aspect of the present invention may be linked through a linking moiety.

Examples of linking moieties include but are not limited to a simple covalent bond, a flexible peptide linker, a disulfide bridge or a polymer such as polyethylene glycol (PEG). Peptide linkers may be entirely artificial (e.g., comprising 2 to 100 amino acid residues independently selected from the group consisting of glycine, serine, asparagine, threonine, proline, valine and alanine) including their natural posttranslational modification e.g. O- and N-glycosylations or adopted from naturally occurring proteins. Disulfide bridge formation can be achieved, e.g., by addition of cysteine residues, as further described herein below. Linking through polyethylene glycols (PEG) can be achieved by reaction of monomers having free cysteines with multifunctional PEGs, such as linear bis-maleimide PEGs. Alternatively, linking can be performed though the glycans on the monomer after their oxidation to aldehyde form and using multifunctional PEGs containing aldehyde-reactive groups.

Selection of the position of the link between the two monomers should take into account that the link should not substantially interfere with the ability of the dimer to bind to ACE2.

Thus, according to one embodiment the linker comprises the amino acid sequence as set forth in SEQ ID NOs: 41 or 42.

Non-peptide linkers are also possible. For example, alkyl linkers such as —NH—(CH2)ₛ-C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker.

According to another embodiment the link is effected using a coupling agent.

The term “coupling agent”, as used herein, refers to a reagent that can catalyze or form a bond between two or more functional groups intra-molecularly, inter-molecularly or both. Coupling agents are widely used to increase polymeric networks and promote crosslinking between polymeric chains, hence, in the context of some embodiments of the present invention, the coupling agent is such that can promote crosslinking between polymeric chains; or such that can promote crosslinking between amino functional groups and carboxylic functional groups, or between other chemically compatible functional groups of polymeric chains. In some embodiments of the present invention the term “coupling agent” may be replaced with the term “crosslinking agent”. In some embodiments, one of the polymers serves as the coupling agent and acts as a crosslinking polymer.

By “chemically compatible” it is meant that two or more types of functional groups can react with one another so as to form a bond.

Exemplary functional groups which are typically present in gelatins and alginates include, but are not limited to, amines (mostly primary amines —NH₂), carboxyls (—CO₂H), sulfhydryls and hydroxyls (—SH and —OH respectively), and carbonyls (—COH aldehydes and —CO— ketones).

Primary amines occur at the N-terminus of polypeptide chains (called the alpha-amine), at the side chain of lysine (Lys, K) residues (the epsilon-amine), as found in gelatin, as well as in various naturally occurring polysaccharides and aminoglycosides. Because of its positive charge at physiologic conditions, primary amines are usually outward-facing (i.e., found on the outer surface) of proteins and other macromolecules; thus, they are usually accessible for conjugation.

Carboxyls occur at the C-terminus of polypeptide chain, at the side chains of aspartic acid (Asp, D) and glutamic acid (Glu, E), as well as in naturally occurring aminoglycosides and polysaccharides such as alginate. Like primary amines, carboxyls are usually on the surface of large polymeric compounds such as proteins and polysaccharides.

Sulfhydryls and hydroxyls occur in the side chain of cysteine (Cys, C) and serine, (Ser, S) respectively. Hydroxyls are abundant in polysaccharides and aminoglycosides.

Carbonyls as ketones or aldehydes can be form in glycoproteins, glycosides and polysaccharides by various oxidizing processes, synthetic and/or natural.

According to some embodiments of the present invention, the coupling agent can be selected according to the type of functional groups and the nature of the crosslinking bond that can be formed therebetween. For example, carboxyl coupling directly to an amine can be afforded using a carbodiimide type coupling agent, such as EDC; amines may be coupled to carboxyls, carbonyls and other reactive functional groups by N-hydroxysuccinimide esters (NHS-esters), imidoester, PFP-ester or hydroxymethyl phosphine; sulfhydryls may be coupled to carboxyls, carbonyls, amines and other reactive functional groups by maleimide, haloacetyl (bromo- or iodo-), pyridyldisulfide and vinyl sulfone; aldehydes as in oxidized carbohydrates, may be coupled to other reactive functional groups with hydrazide; and hydroxyl may be coupled to carboxyls, carbonyls, amines and other reactive functional groups with isocyanate.

Hence, suitable coupling agents that can be used in some embodiments of the present invention include, but are not limited to, carbodiimides, NHS-esters, imidoesters, PFP-esters or hydroxymethyl phosphines.

As mentioned, the polypeptides described herein are attached to the outer surface of particles.

Exemplary particles that may be used according to this aspect of the present invention include, but are not limited to polymeric particles, microcapsules, liposomes, microspheres, microemulsions, nanoparticles, nanocapsules, nano-spheres, nano-liposomes, nano-emulsions and nanotubes.

According to a particular embodiment, the particles are nanoparticles.

As used herein, the term “nanoparticle” refers to a particle or particles having an intermediate size between individual atoms and macroscopic bulk solids. Generally, nanoparticle has a characteristic size (e.g., diameter for generally spherical nanoparticles, or length for generally elongated nanoparticles) in the sub-micrometer range, e.g., from about 1 nm to about 500 nm, or from about 1 nm to about 200 nm, or of the order of 10 nm, e.g., from about 1 nm to about 100 nm. The nanoparticles may be of any shape, including, without limitation, elongated particle shapes, such as nanowires, or irregular shapes, in addition to more regular shapes, such as generally spherical, hexagonal and cubic nanoparticles. According to one embodiment, the nanoparticles are generally spherical.

The particles of this aspect of the present invention may have a charged surface (i.e., positively charged or negatively charged) or a neutral surface.

Agents which are used to fabricate the particles may be selected according to the desired charge required on the outer surface of the particles.

Thus, for example if a negatively charged surface is desired, the particles may be fabricated from negatively charged lipids (i.e. anionic phospholipids) such as described herein below.

When a positively charged surface is desired, the particles may be fabricated from positively charged lipids (i.e. cationic phospholipids), such as described herein below.

Non-charged particles are also contemplated by the present invention. Such particles may be fabricated from neutral lipids such as phosphatidylethanolamine or dioleilphosphatidylethanolamine (DOPE).

It will be appreciated that combinations of different lipids may be used to fabricate the particles of the present invention, including a mixture of more than one cationic lipid, a mixture of more than one anionic lipid, a mixture of more than one neutral lipid, a mixture of at least one cationic lipid and at least one anionic lipid, a mixture of at least one cationic lipid and at least one neutral lipid, a mixture of at least one anionic lipid and at least one neutral lipid and additional combinations of the above. In addition, polymer-lipid based formulations may be used.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactie-polyglycolic acid’ polyvinyl alcohol, polyvinylpyrrolidone, polymethoxazoline, polyethyloxazoline, polyllydroxyetlyloxazolille, solyhydroxypryloxazoline, polyaspartarllide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose.

The polymers may be employed as homopolymers or as block or random copolymers.

The particles may also include other components. Examples of such other components includes, without being limited thereto, fatty alcohols, fatty acids, and/or cholesterol esters or any other pharmaceutically acceptable excipients which may affect the surface charge, the membrane fluidity and assist in the incorporation of the biologically active lipid into the lipid assembly. Examples of sterols include cholesterol, cholesterol hemisuccinate, cholesterol sulfate, or any other derivatives of cholesterol. Preferred lipid assemblies according the invention include either those which form a micelle (typically when the assembly is absent from a lipid matrix) or those which form a liposome (typically, when a lipid matrix is present).

In one embodiment, the particle is a lipid-based nanoparticle. The core of the particle may be hydrophilic or hydrophobic. The core of the lipid-based nanoparticle may comprise some lipids, such that it is not fully hydrophilic.

In a specific embodiment, the particle is a liposome. As used herein and as recognized in the art, liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September;64(1-3):35-43].

The liposomes may be unilamellar or may be multilamellar. Unilamellar liposomes may be preferred in some instances as they represent a larger surface area per lipid mass. Suitable liposomes in accordance with the invention are preferably non-toxic. The liposomes may be fabricated from a single phospholipid or mixtures of phospholipids. The liposomes may also comprise other lipid materials such as cholesterol. For fabricating liposomes with a negative electrical surface potential, acidic phospho- or sphingo- or other synthetic-lipids may be used. Preferably, the lipids have a high partition coefficient into lipid bilayers and a low desorption rate from the lipid assembly. Exemplary phospholipids that may be used for fabricating liposomes with a negative electrical surface potential include, but are not limited to phosphatidylserine, phosphatidic acid, phosphatidylcholine and phosphatidyl glycerol.

Other negatively charged lipids which are not liposome forming lipids that may be used are sphingolipids such as cerebroside sulfate, and various gangliosides.

The most commonly used and commercially available lipids derivatized into lipopolymers are those based on phosphatidyl ethanolamine (PE), usually distearylphosphatidylethanolamine (DSPE).

The lipid phase of the liposome may comprise a physiologically acceptable liposome forming lipid or a combination of physiologically acceptable liposome forming lipids for medical or veterinarian applications. Liposome-forming lipids are typically those having a glycerol backbone wherein at least one of the hydrofoil groups is substituted with an acyl chain, a phosphate group, a combination or derivatives of same and may contain a chemically reactive group (such as an as amine imine, acids ester, aldelhyde or alcohol) at the headgroup. Typically, the acyl chain is between 12 to about 24 carbon atoms in length, and has varying degrees of saturation being fully, partially or non-hydrogenated lipids. Further, the lipid matrix may be of natural source, semi-synthetic or fully synthetic lipid, and neutral, negatively or positively charged.

According to one embodiment, the lipid phase comprises phospholipids.

The phospholipids may be a glycerophospholipid. Examples of glycerophospholipid include, without being limited thereto, phosphatidylglycerol (PG) including dimyristoyl phosphatidylglycerol (DMPG); phosphatidylcholine (PC), including egg yolk phosphatidylcholine and dimyristoyl phosphatidylcholine (DMPC), phosphatidic acid (PA), phosphatidylinositol (PI), phosphatidylserine (PS) and sphingomyelin (SM) and derivatives of the same.

Another group of lipid matrix employed according to the invention includes cationic lipids (monocationic or polycationic lipids). Cationic lipids typically consist of a lipophilic moiety, such as a sterol or the same glycerol backbone to which two acyl or two alkyl, or one acyl and one alkyl chain contribute the hydrophobic region of the amphipathic molecule, to form a lipid having an overall net positive charge.

Preferably, the head groups of the lipid carries the positive charge. Monocationic lipids may include, for example, 1,2-dimyristoyl-3-trimethylammonium propane (DMTAP) 1,2-dioleyloxy-3-(trimethylanino) propane (DOTAP), N-[−1-(2,3,-ditetradecyloxy)propyl]-N,N-dimethyl-N-hydroxyethylammonium bromide (DMRIE), N-[1-(2,3,-dioleyloxy)propyl]-N,N-dimethyl-N-hydroxy ethyl-ammonium bromide (DORIE), N-[1-(2,3-dioleyloxy) propyl]; —N,N,N-trimethylammonium chloride (DOTMA); 3;N—(N′,N′-dimethylaminoethane) carbamoly]; cholesterol (DC-Chol), and I dimethyl-dioctadecylammonium (DDAB).

Examples of polycationic lipids include a similar lipoplilic moiety as with the mono cationic lipids, to which spermine or spermidine is attached. These include’ without being limited thereto, N-[2-[[2,5-bis[3-aminopropyl)amino]-1-oxopentyl]amino]ethyl]N,N dimethul-2,3 bis (1-oXo-9-octadecenyl) oXy];-1 propanaminium (DOSPA), and ceramide carbamoyl spermine (CCS).

The cationic lipids may be used alone, in combination with cholesterol, with neutral phospholipids or other known lipid assembly components. In addition, the cationic lipids may form part of a derivatized phospholipids such as the neutral lipid dioleoylphosphatidyl ethanolamine (DOPE) derivatized with polylysine to form a cationic lipopolymer.

The diameter of the liposomes used preferably ranges from 50-200 nM and more preferably from 20-100 nM. For sizing liposomes, extrusion, homogenization or exposure to ultrasound irradiation may be used, Homogenizers which may be conveniently used include microfluidizers produced by Microfluidics of Boston, MA. In a typical homogenization procedure, liposomes are recirculated through a standard emulsion homogenizer until selected liposomes sizes are observed. The particle size distribution can be monitored by conventional laser beam particle size discrimination. Extrusion of liposomes through a small-pore polycarbonate membrane or an asymmetric ceramic membrane is an effective method for reducing liposome sizes to a relatively well defined size distribution. Typically, the suspension is cycled through the membrane one or more times until the desired liposome size distribution is achieved. The liposomes may be extruded through successively smaller pore membranes to achieve a gradual reduction in liposome size.

The particles of the present invention may be modified. According to one embodiment, the particles are modified to enhance their circulatory half-life (e.g. by PEGylation) to reduce their clearance, to prolong their scavenging time-frame and to allow antibody binding. The PEG which is incorporated into the articles may be characterized by of any of various combinations of chemical composition and/or molecular weight, depending on the application and purpose.

According to a particular embodiment, the particles are coupled to vitamin A—e.g. vitamin A coated liposomes—see for e.g. Sato et al., Nat Biotechnol. 2008 April; 26(4):431-42.

The particle may comprise a vesicle. The particle may comprise an exosome. The particles described here may comprise any one or more of the properties of the exosomes described herein.

The particle may comprise a vesicle or a flattened sphere limited by a lipid bilayer. The particles may comprise diameters of 40-100 nm. The particles may be formed by inward budding of the endosomal membrane. The particles may have a density of .about. 1.13-1.19 g/ml and may float on sucrose gradients. The particles may be enriched in cholesterol and sphingomyelin, and lipid raft markers such as GM1, GM3, flotillin and the src protein kinase Lyn.

According to a particular embodiment, the particle is an exosome.

As used herein, the term “exosome” refers to an extracellular vesicle that is released from a cell upon fusion of a multivesicular body (MVB) with the plasma membrane.

The exosome may (a) have a size of between 50 nm and 300 nm as determined by electron microscopy; (b) comprise a complex of molecular weight >100 kDa, comprising proteins of <100 kDa; (c) comprise a complex of molecular weight >300 kDa, comprising proteins of <300 kDa; (d) comprise a complex of molecular weight >1000 kDa; (e) have a size of between 2 nm and 200 nm, as determined by filtration against a 0.2 μM filter and concentration against a membrane with a molecular weight cut-off of 10 kDa; or (f) have a hydrodynamic radius of below 100 nm, as determined by laser diffraction or dynamic light scattering.

The particle may be isolated from cells which express ACE2 on their surface or a conditioned medium of said cells. The particle may be produced or isolated in a number of ways. Such a method may comprise isolating the particle from cells which express ACE2 on their surface. Such a method may comprise isolating the particle from a conditioned medium.

The particle may be isolated for example by being separated from non-associated components based on any property of the particle. For example, the particle may be isolated based on molecular weight, size, shape, composition or biological activity.

The conditioned medium may be filtered or concentrated or both during, prior to or subsequent to separation. For example, it may be filtered through a membrane, for example one with a size or molecular weight cut-off. It may be subject to tangential force filtration or ultrafiltration.

For example, filtration with a membrane of a suitable molecular weight or size cutoff, as described in the Assays for Molecular Weight elsewhere in this document, may be used.

The conditioned medium, optionally filtered or concentrated or both, may be subject to further separation means, such as column chromatography. For example, high performance liquid chromatography (HPLC) with various columns may be used. The columns may be size exclusion columns or binding columns.

One or more properties or biological activities of the particle may be used to track its activity during fractionation of the HC-CM. As an example, light scattering, refractive index, dynamic light scattering or UV-visible detectors may be used to follow the particles. For example, a therapeutic activity may be used to track the activity during fractionation.

The particle may have a size of greater than 2 nm. The particle may have a size of greater than 5 nm, 10 nm, 20 nm, 30 nm, 40 nm or 50 nm. The particle may have a size of greater than 100 nm, such as greater than 150 nm. The particle may have a size of substantially 200 nm or greater.

The particle or particles may have a range of sizes, such as between 2 nm to 20 nm, 2 nm to 50 nm, 2 nm to 100 nm, 2 nm to 150 nm or 2 nm to 200 nm. The particle or particles may have a size between 20 nm to 50 nm, 20 nm to 100 nm, 20 nm to 150 nm or 20 nm to 200 nm. The particle or particles may have a size between 50 nm to 100 nm, 50 nm to 150 nm or 50 nm to 200 nm. The particle or particles may have a size between 100 nm to 150 nm or 100 nm to 200 nm. The particle or particles may have a size between 150 nm to 200 nm.

The size may be determined by various means. In principle, the size may be determined by size fractionation and filtration through a membrane with the relevant size cut-off. The particle size may then be determined by tracking segregation of component proteins with SDS-PAGE or by a biological assay.

The size may comprise a hydrodynamic radius. The hydrodynamic radius of the particle may be below 100 nm. It may be between about 30 nm and about 70 nm. The hydrodynamic radius may be between about 40 nm and about 60 nm, such as between about 45 nm and about 55 nm. The hydrodynamic radius may be about 50 nm.

The hydrodynamic radius of the particle may be determined by any suitable means, for example, laser diffraction or dynamic light scattering.

Methods of coupling affinity the ACE-2 affinity moieties on particle's outer surface (e.g., liposomes) are known in the art.

As used herein “coupling” or “coupled on” refers to covalent or non-covalent attachment of the affinity moiety to the particle.

Conjugation methods which can be used in accordance with the teachings of the present invention can be divided to direct binding or indirect binding. Some methods are provided hereinbelow and are summarized in Ansell, Supra. While specifically referring to liposomes, the procedures described hereinbelow may be applied to a variety of particles, while using modified protocols simply applied by the ordinary artisan.

Direct conjugation methods are well known to those of skill in the art. See for example, G. Gregoriadis, (1984) “Liposome Technology” CRC Press, Boca Raton, Fla. and D. D. Lasic, “Liposomes: from physics to applications” (1993) Elsevier, Amsterdam; N.Y Particularly preferred is conjugation through a thioether linkage. This may be accomplished by reacting the ACE-2 affinity moiety with a maleimide derivatized lipid such as maleimide derivatized phosphatidylethanolamine (M-PE) or dipalmitoylethanolamine (M-DEP). This approach is described in detail by Martin et al. J. Biol. Chem., 257: 286-288 (1982) which is incorporated herein by reference.

In another preferred embodiment, the ACE-2 affinity moiety can be coupled to a hydrophilic polymer (e.g., a PEG). Means of attaching targeting molecules to polymer linkers are well known to those of skill in the art (see, e.g., chapter 4 in Monoclonal Antibodies: Principles and Applications, Birch and Lennox, eds., John Wiley & Sons, Inc., New York (1995); and Blume et al. Biochem. Biophys. Acta. 1149: 180-184 (1993). In one embodiment, the ACE-2 affinity moiety is linked to a maleimide derivatized PEG through the —SII group of the moiety. The maleimide-derivative of PEG-PE is included in the liposome preparation as described above and below and the ACE-2 affinity moiety can be conjugated with the liposome via the sulfhydryl group at pH 7.2.

Amine modifications making use of cross-linking agents such as EDC are taught in Endoh et al. 1981 J. Immun. Meth. 44:79-85; Dunnick 1975 J. Nuclear. Med. 16:483-487; Alternatively, direct modification of the ACE-2 affinity moiety with activated fatty acids, such as N-hydroxysuccinimide (NHS) eater or palmitic acid, prior to incorporation into a liposome membrane, typically by detergent dialysis procedures (Huang et al. 1980, J. Biol. Chem. 255:8015-8018. Reagents, such as EDC, are used in conjunction with NHS to activate acidic functions on liposomes, which are then conjugated to the amino groups on ACE-2 affinity moieties. Better control of the conjugation reaction can be achieved using heterobifunctional cross-linkers which efficiently introduce a unique and selective reactive function, such as a protected thiol or maleimide group. Examples of these crosslinkers are SPDP (Barbet et al. 1981 J. Supramolec. Struct. Cell. Biochem. 16:243-258), S-acetylthioglycolic acid N-hydroxysuccinimide ester (SATA, Jones 1993 Biochim. Biophys. Acta. 1152:23:1-32; Schwendener 1990 Biochim. Biophys. Acta. 1026:69-79 and 4-(p-maleimidophenyl)butyric acid N-hydroxysuccinimide ester (SMPB (Hansen 1995 Biochim. Biophys. Acta. 1239:133-144). ACE-2 affinity moieties which have been activated by these crosslinkers can, after deprotection where appropriate, react with activated lip-ids in liposome bilayers. Maleimide and protected thiol-derivatized lipids are available from commercial sources for this purpose.

Deprotection of 3-pyridyl disulfides is usually effected by DTT and occasionally by some other mercaptan. Once deprotected, sulfhydryl groups can react with maleimide (for example SMPB-modified conjugates) or iodo (for example, iodoacetic acid N-hydroxysuccinimide ester (SIAA)-modified conjugates) groups. Maleimide groups are recommended since iodo functions can react with amino groups in either of the substrates, leading to undesirable side products. Deprotection is not required for these reagents.

Indirect Conjugation Methods:

Biotin-avidin—For example, a biotin conjugated ACE-2 affinity moiety may be bound to a particle (e.g., liposome) containing a streptavidin. Alternatively, the biotinylated ACE-2 affinity moiety may be conjugated to a biotin derivatized liposome by an avidin or streptavidin linker. Ahmad et al., Cancer Res., 52: 4817-4820 (1992) which is herein incorporated by reference, describes such a mode of coupling.

Loading of the particle with the pharmaceutical agent can be effected concomitant with, or following particle assembly.

When the pharmaceutical agent of interest is a nucleic acid, e.g., DNA, RNA, siRNA, plasmid DNA, short-hairpin RNA, small temporal RNA (stRNA), microRNA (miRNA), RNA mimetics, or heterochromatic siRNA, the nucleic acid agent of interest has a charged backbone that prevents efficient encapsulation in the lipid particle. Accordingly, the nucleic acid agent of interest may be condensed with a cationic polymer, e.g., PEI, polyamine spermidine, and spermine, or cationic peptide, e.g., protamine and polylysine, prior to encapsulation in the lipid particle. In one embodiment, the agent is not condensed with a cationic polymer.

In another embodiment, the particle disclosed herein is used for delivery of the pharmaceutical agent of interest encapsulated therein.

In another embodiment, the RBD-62 tagged EV, also termed “EVsRBD-62”, mediates delivery of a pharmaceutical agent of interest into target tissues having ACE2 receptor, through binding of the RBD-62 tagged EV to ACE2 receptors.

In another embodiment, the pharmaceutical agent of interest is encapsulated in the lipid particle in the following manner. The particle is provided lyophilized. The agent of interest is in an aqueous solution. The agent of interest in aqueous solution is utilized to rehydrate the lyophilized lipid particle. Thus, the agent of interest is encapsulated in the rehydrated lipid particle.

In one embodiment, two agents of interest may be delivered by the particles (e.g., lipid based particle). One agent is hydrophobic and the other is hydrophilic. The hydrophobic agent may be added to the lipid particle during formation of the lipid particle. The hydrophobic agent associates with the lipid portion of the lipid particle. The hydrophilic agent is added in the aqueous solution rehydrating the lyophilized lipid particle. In an exemplary embodiment of two agent delivery a condensed siRNA is encapsulated in a liposome and wherein a drug that is poorly soluble in aqueous solution is associated with the lipid portion of the lipid particle. As used herein, “poorly soluble in aqueous solution” refers to a composition that is less that 10% soluble in water.

As used herein “loading” refers to encapsulating or absorbing.

The term “encapsulated” as used herein refers to the pharmaceutical agent being distributed in the interior portion of the particles. Preferably, the pharmaceutical agents are homogenously distributed. Homogeneous distribution of a pharmaceutical agent in polymer particles is known as a matrix encapsulation. However, due to the manufacturing process it is foreseen that minor amounts of the pharmaceutical agent may also be present on the outside of the particle and/or mixed with the polymer making up the shell of the particle.

As used herein “absorbed” refers to binding of the pharmaceutical agent to the outer surface of the particle.

Agents which may be targeted to ACE-2 expressing cells include but are not limited to therapeutic agents and diagnostic agents.

Exemplary therapeutic agents include nucleic acid, polypeptides e.g. antibodies, anticancer agent (e.g., chemotherapy, radioisotopes, immunotherapy), antibiotic, enzyme, antioxidant, lipid intake inhibitor, hormone, anti-inflammatory, steroid, vasodilator, angiotensin converting enzyme inhibitor, angiotensin receptor antagonist, inhibitor for smooth muscle cell growth and migration, platelet aggregation inhibitor, anticoagulant, inhibitor for release of chemical mediator, promoter or inhibitor for endothelial cell growth, aldose reductase inhibitor, inhibitor for mesangium cell growth, lipoxygenase inhibitor, immunosuppressive, immunostimulant, antiviral agent, Maillard reaction suppressor, amyloidosis inhibitor, nitric oxide synthetic inhibitor, AGEs (Advanced glycation end-products) inhibitor, radical scavenger, protein, peptide; glycosaminoglycan and derivatives thereof; and oligosaccharide, polysaccharide, and derivatives thereof.

According to a particular embodiment, the pharmaceutical agent is a cytotoxic agent.

As used herein, the term “cytotoxic agent” refers to refers to a substance that inhibits or prevents the function of cells and/or causes destruction of cells. The term is intended to include radioactive isotopes (e.g., ²¹¹At, ¹³¹I, ¹²⁵I, ³²P, ³⁵S and radioactive isotopes of Lu, including ¹⁷⁷Lu, ⁸⁶Y, ⁹⁰Y, ¹¹¹In, ¹⁷⁷Lu, ²²⁵Ac, ²¹²Bi, ²¹³Bi, ⁶⁶Ga, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁴⁸V, ²⁰³Pb, ²⁰⁹Pb, ²¹²Pb, ¹⁶⁶Ho, ¹⁴⁹Pm, ¹⁵³Sm, ²⁰¹Tl, ¹⁸⁸Re, ¹⁸⁶Re and ⁹⁹mTc), anticancer agents as otherwise described herein, including chemotherapeutic (anticancer drugs e.g. methotrexate, adriamicin, vinca alkaloids (vincristine, vinblastine, etoposide), taxol, doxoruicin, cisplatin, 5-fluorouridine, melphalan, ethidium bromide, mitomycin C, chlorambucil, daunorubicin and other intercalating agents, enzymes and fragments thereof such as nucleolytic enzymes, antibiotics, therapeutic RNA molecules (e.g., siRNA, antisense oligonucleotides, microRNA, ribozymes, RNA decoys, aptamers), DNAzymes, antibodies, proteins and polynucleotides encoding same, and toxins such as small molecule toxins or enzymatically active toxins of bacterial, fungal, plant or animal origin, such as pokeweed antiviral protein (PAP), ricin toxin A, abrin, gelonin, saporin, cholera toxin A, diphtheria toxin, Pseudomonas exotoxin, and alpha-sarcin, including fragments and/or variants thereof.

As mentioned, the present invention contemplates the use of RNA silencing agents as pharmaceutical agents. The RNA silencing agents may be directed against any protein depending on the disease being treated.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates encapsulation of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the present invention also contemplates encapsulation of long dsRNA (over 30 base transcripts) for gene silencing in cells where the interferon pathway is not activated (e.g. embryonic cells and oocytes) see for example Billy et al., PNAS 2001, Vol 98, pages 14428-14433 and Diallo et al, Oligonucleotides, Oct. 1, 2003, 13(5): 381-392. doi:10.1089/154545703322617069.

The term “siRNA” refers to small inhibitory RNA duplexes (generally between 18-30 basepairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27mer) instead of a product (21 mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

It will be appreciated that more than one siRNA agent may be used to down-regulate a target gene.

The strands of a double-stranded interfering RNA (e.g., a siRNA) may be connected to form a hairpin or stem-loop structure (e.g., a shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to a miRNA, rather than triggering RNA degradation.

It will be appreciated that the RNA silencing agent of the present invention need not be limited to those molecules containing only RNA, but further encompasses chemically-modified nucleotides and non-nucleotides.

According to yet another embodiment, the therapeutic agent is an anti-viral agent.

In particular, the anti-viral agent may be one used for treating a respiratory viral infection.

Specific examples for drugs that are routinely used for the treatment of respiratory viral infections (such as COVID-19) include, but are not limited to, Lopinavir/Ritonavir, Nucleoside analogues, Neuraminidase inhibitors, Remdesivir, polypeptide (EK1), abidol, RNA synthesis inhibitors (such as TDF, 3TC), anti-inflammatory drugs (such as hormones and other molecules), According to a specific embodiment, the anti-inflammatory agent is interferon I.

According to a specific embodiment, the interferon I is IFNβ and/or IFNα2.

Exemplary diagnostic agents that can be incorporated in the particles of the invention include those that comprise radioactive isotope (such as ^{[125]}iodine), phosphorescent chemicals, chemiluminescent chemical a fluorescent chemicals (fluorophore), enzymes, fluorescent polypeptides, molecules (contrast agents) detectable by Positron Emission Tomagraphy (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Fluorescence detection methods which can be used to detect the antibody when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

As mentioned, the particles may be used for treating or diagnosing a disease. It will be appreciated that selection of the particular pharmaceutical agent comprised within/on the particles is dependent on the disease being treated/diagnosed. Thus, for example when the disease being treated is cancer, the agent comprised in the particles is an anti-cancer agent (e.g. a cytotoxic agent). When the disease being treated is a respiratory viral infection, the agent comprised in the particles is an anti-viral agent.

Thus, according to another aspect of the present invention there is provided a method of treating an ACE2-associated disease in a subject in need thereof, comprising administering to the subject a therapeutically effective amount of a particle having an ACE2 targeting moiety attached to an outer surface thereof, wherein said ACE2 targeting moiety comprises a polypeptide comprising an amino acid sequence of SARS CoV-2 receptor-binding domain (RBD), wherein said amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein said polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein said polypeptide comprises at least 170 amino acids of said RBD, wherein said particle is attached to or encapsulates a therapeutic agent, thereby treating the disease.

According to still another aspect of the present invention, there is provided a method of diagnosing an ACE2-associated disease in a subject in need thereof, comprising administering to the subject an effective amount of a particle having an ACE2 targeting moiety attached to an outer surface thereof, wherein said ACE2 targeting moiety comprises a polypeptide comprising an amino acid sequence of SARS CoV-2 receptor-binding domain (RBD), wherein said amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein said polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein said polypeptide comprises at least 170 amino acids of said RBD, wherein said particle is attached to or encapsulates a diagnostic agent, thereby diagnosing the disease.

According to a particular embodiment, the disease being treated/diagnosed in a respiratory disease.

Contemplated respiratory diseases include respiratory infections, inflammatory respiratory diseases, such as asthma, COPD and chronic bronchitis; genetic diseases such as cystic fibrosis; allergic conditions (atopy, allergic inflammation); bronchiectasis.

The respiratory infection may be bacterial or viral.

Examples of contemplated respiratory infection include coronaviral infections, rhinoviral infections, pneumonia, rihinitis and influenza infection.

The coronaviral infection may be caused by one of the betacoronaviruses such as severe acute respiratory syndrome coronaviruses (SARS-CoV or, SARS-CoV-2 and Middle East respiratory syndrome coronavirus (MERS-CoV).

As used herein, “Coronavirus” refers to enveloped positive-stranded RNA viruses that belong to the family Coronaviridae and the order Nidovirales.

Examples of Corona viruses which are contemplated herein include, but are not limited to, 229E, NL63, OC43, and HKU1 with the first two classified as antigenic group 1 and the latter two belonging to group 2, typically leading to an upper respiratory tract infection manifested by common cold symptoms.

However, coronaviruses, which are zoonotic in origin, can evolve into a strain that can infect human beings leading to fatal illness. Thus particular examples of Coronaviruses contemplated herein are SARS-CoV, Middle East respiratory syndrome Coronavirus (MERS-CoV), and the recently identified SARS-CoV-2 [causing 2019-nCoV (also referred to as “COVID-19”)].

It would be appreciated that any coronavirus strain is contemplated herein even though SARS-CoV-2 is emphasized in a detailed manner.

According to specific embodiments, the Corona virus is SARS-CoV-2.

Exemplary strains of SARS-CoV-2 which may be effectively treated include, but are not limited to the Wuhan strain, B.1.1.7, B.1.351 and P.1.

According to another embodiment, the disease to be treated is cancer.

In one embodiment, the cancer is lung cancer.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings, male or female, at any age or gender, which suffer from the pathology. Preferably, this term encompasses individuals who are at risk to develop the pathology (e.g., above 65 of age).

The particles of the present invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the loaded particles accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intrapulmonary or intraocular injections.

Alternately, one may administer the pharmaceutical composition in a local rather than systemic manner, for example, via inhalation into the lungs of the subject.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (composition of matter comprising the isolated polypeptides or fusion proteins) effective to prevent, alleviate or ameliorate symptoms of a disorder (e.g., Coronaviral infection) or prolong the survival of the subject being treated.

According to an embodiment of the present invention, an effective amount of the loaded particles of the present invention is an amount selected to neutralize Coronaviruses and/or prevent coronaviruses from entering the lung cells.

Determination of a therapeutically effective amount is well within the capability of those skilled in the art, especially in light of the detailed disclosure provided herein.

For example, any in vivo or in vitro method of evaluating Coronavirus viral load may be employed.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Materials and Methods

Cell culture and transfection: HEK293, HEK293T, ACE2-expressing HEK293T cells, and stable pAGDisplay-RBD cell lines were cultured at 37° C. in a 5% CO₂ atmosphere in DMEM medium (4.5 g/L glucose, L-glutamine; Gibco, USA) supplemented with 10% fetal bovine serum (FBS) and 1% glutamine (4 mM). Cells were passaged 2-3 times per week using trypsin EDTA solution A (Biological Industries, USA) for cell detachment. The stably expressing ACE2 HEK293T cell line was kindly obtained from the lab of Dr. Ron Diskin (Weizmann Institute of Science) and kept under puromycin antibiotics (0.5 μg/ml, Invitrogen, USA).

HEK-pAGDisplay-RBD stable cell line generation: The pAGDisplay-based plasmids expressing RBD (1 ug of DNA) were transfected in a 60 mm culture dish with 80% confluent HEK293 cells by a JetPrime transfection reagent (Polyplus, France) according to the manufacturer's protocol. After transfection (24 h), cells were transferred to a 150 mm culture dish. Subsequently, 48 h after transfection, the media was replaced by fresh DMEM medium supplemented with 10% FBS and 1 μg/ml puromycin (Invitrogen, USA). Puromycin-resistant cells were selected for one week with regular replacement of cell media. Stably transfected cells associated with the top 1% green fluorescence signals (Puro-eUnaG2), were sorted out from the population using the S3e Cell Sorter device (Bio-Rad, USA) and further sub-cultured to single colonies.

Characterization of parental cells: The presence of the RBD on the cell surface was measured by fluorescence-activated cell sorting (FACS). The cells were cultured until full confluence, then they were detached from the plates by PBS and added to the Eppendorf tubes. After spinning down (5 min at 500 g), the cell pellet was resuspended in a labeling solution containing fluorescently labeled, purified ACE2 protein in PBS supplemented with 2% FBS. The cells were labeled for 1h on ice and then washed two times with 1 mL of PBS (spun down each time for 5 min at 500 g). After the last wash, the cells were resuspended in PBS with 2% FBS solution, filtered by 0.45 m filters, and added to the FACS tubes. The fluorescence was measured by the LRSII FACS machine (BD Biosciences, USA) and analyzed by the FlowJo software.

Preparation of cell ghost vesicles: Vesicles composed of plasma membranes of cell ghost were prepared as previously described (Goh, W. J., S, Z., Czarny, B. & Pastorin, G. nCVTs: a hybrid smart tumour targeting platform. Nanoscale 10, 6812 (2018)), with some modifications. Briefly, approximately 1×10⁷ RBD cells were centrifuged at 500 g for 10 minutes, washed once with phosphate buffered saline (PBS) and suspended in 0.06% w/v sucrose in 0.25×PBS, supplemented with 1% v/v penicillin streptomycin antibiotics and 0.5% v/v protease inhibitor cocktail, on a shaker overnight at room temperature. The resulting cell suspension was subsequently centrifuged at 6000 g for 10 minutes and suspended in 0.06% w/v sucrose in 1×PBS on a shaker overnight at room temperature. Subsequently, the cell suspension was centrifuged again at 6000 g for 10 minutes and suspended in PBS−/−, and extruded sequentially through 10, 5, 1 and 0.1 μm polycarbonate membrane filters (Whatman) using a mini extruder (Avanti Polar Lipids) 5 times each filter.

Preparation of large unilamellar vesicles (LUVs): LUVs were prepared composed of DOPC. Lipid solution in chloroform was placed in a glass vial, and the organic solvent was evaporated by 12 h of vacuum pumping. The lipid film was then hydrated with PBS−/− to reach the desired concentration and gently vortexed. The resulting MLV suspension was then sonicated for 10 min to disperse larger aggregates and the liposomal suspension was extruded 21 times through polycarbonate filters (100 nm pore size, Avanti Polar Lipids) using a mini-extruder (Avanti Polar Lipids). Size and concentration were verified using NTA and the liposomal suspension was used within 2 weeks from extrusion. The extruded sample is then pipetted on the top of a 10%-50% Optiprep band and ultracentrifuged at 100000 g for 2 h at 4° C. using a SW41 rotor. The ghost vesicles are then collected from the interface of the gradient and utilized for further experiments.

EV isolation: EV-depleted medium was prepared by two rounds of ultracentrifugation (100,000 g, 16 h) of DMEM with 20% FBS, diluted to 10% FBS and supplemented with glutamine. For EVs isolation, cells were cultured in EV-free medium for 48-72 h, then the medium was collected and processed by differential centrifugation (400 g, 10 min; 2000 g, 10 min; 10,000 g, 30 min, all at 4° C.). The final supernatant was collected and EVs were pelleted at 100,000 g at 4° C. for four hours in an Optima ultracentrifuge using the Beckman Ti45 rotor (Beckman Coulter, USA). The EVs pellet was washed with PBS and resuspended in 0.22 m filtered PBS. EVs were isolated from the RBD-transfected cells (EV^{RBD} and EV^{RBD62}) and control cells (EV^{noRBD})

EV NTA analysis: The size and concentration of EVs diluted in PBS (1:1000 or 1:5000) was measured by nanoparticle tracking analysis using the NanoSight system (Malvern Instruments, UK) with a 405 nm laser by acquiring five, one-minute videos at the camera level 16. Threshold 5 was used for the analysis in all samples. Protein content was measured by the BCA (cells) or microBCA (EVs) protein assay according to the manufacturer's instructions (Sigma Aldrich, USA).

In Vitro Binding of EVs^{RBD} and EVs^{RBD62} to the ACE2 Receptor:

Flow cytometry: To confirm binding and uptake of EV^{RBD} in ACE2-expressing cells, isolated EVs^{RBD}, EVs^{RBD62} or EV^{noRBD} were fluorescently labeled with 1,1′-Dioctadecyl-3,3,3′,3′-Tetramethylindotricarbocyanine Iodide (DiR) (ThermoFischer Scientific, D12731) at a concentration of 15 μM by incubation for 1h at room temperature. EVs were then washed three times with the VivaSpin centrifugation filters—five min at 14 000 g, and two min at 1000 g in a reverse position. HEK293T or ACE-expressing HEK293T cells were incubated with labeled EVs^{RBD} or EV^{noRBD} in EV-depleted DMEM for one or three h. Then, the cells were detached from the plate by PBS, washed twice with PBS in the Eppendorf tubes (spun down five min at 500 g), filtered by 0.45 m filters, and added to the FACS tubes in FACS solution (PBS with 2% FBS). The fluorescence signal (APC-Cy7 filter) of cells was measured using a LSRII cell analyzer flow cytometer (BD Biosciences) and analyzed by the FlowJo software.

Fluorescence microscopy: For visualization by fluorescence microscopy, cells were seeded on 14 mm-diameter coverslips in a 24-well plate. The wells were coated with fibronectin by incubation for 45 min. Then, fibronectin was removed and the wells were washed with PBS before seeding the cells. For uptake of EVs in cells, the DiD-labeled EVs were dissolved in exosome-free medium and incubated with cells for three hours. Then, the medium was removed, and cells were washed two times with PBS. For staining of cell nuclei, DAPI was dissolved in 2.5% formaldehyde and the cells were incubated in the solution for 20 min. After two washes, the coverslips were carefully transferred and mounted on glass slides and the fluorescence was visualized using a wide-field microscope (Leica DMI8).

Results

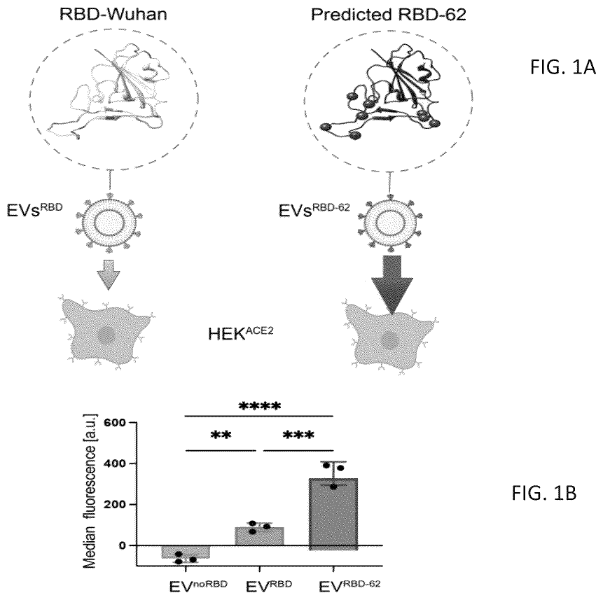

EVs were engineered to express the predicted “Wuhan variant” SARS-CoV-2 RBD and the “62” variant of SARS-CoV-2 RBD (FIGS. 1A-B). To this end, the gene encoding for the Wuhan variant RBD and the RBD sequence of the predicted “62” variant of SARS-CoV were designed and cloned into a pAGDisplay and HEK-293 cells were transfected to obtain a stable cell (HEK^{RBD-62} and HEK^{RBD}). After EV isolation, the difference in the binding capabilities of EVs^{RBD} to EVs^{RBD-62} to ACE2 receptors expressed at the surface of live cells was examined using flow cytometry. As shown in FIGS. 1A-B, HEK^{ACE2} Cells incubated with engineered EVs^{RBD-62} showed a four times higher fluorescence compared to that obtained when the same cells were incubated with EVs^{RBD} (the so-called Wuhan variant).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A particle having an ACE2 targeting moiety attached to an outer surface thereof, wherein said ACE2 targeting moiety comprises a polypeptide comprising an amino acid sequence of SARS CoV-2 receptor-binding domain (RBD), wherein said amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein said polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein said polypeptide comprises at least 170 amino acids of said RBD.

2. The particle of claim 1, wherein said ACE2 targeting moiety binds to ACE2 receptors on lung cells with at least 2 fold higher affinity than the wild-type RBD having an amino acid sequence as set forth in SEQ ID NO: 45, when assayed under identical conditions.

3. The particle of claim 1, being attached to, or encapsulating, a therapeutic agent or a diagnostic agent.

4. The particle of claim 3, wherein said therapeutic agent is selected from the group consisting of an antiviral agent, a cytotoxic agent, a bronchodilator, an antibiotic and an anti-inflammatory agent.

5. The particle of claim 3, wherein said therapeutic agent or diagnostic agent is selected from the group consisting of a polynucleotide agent, a polypeptide agent and a small molecule agent.

6. The particle of claim 1, being an extracellular vesicle (EV).

7. The particle of claim 1, being a synthetic particle.

8. The particle of claim 1, wherein said polypeptide comprises modifications at each of the positions 358, 484, 498 and 501, and optionally comprising a modification at position 460.

9. The particle of claim 1, wherein said modification at position 358 comprises a I358F substitution, wherein said modification at position 484 comprises a E498K substitution, wherein said modification at position 498 comprises a Q498R substitution, or said modification at position 501 comprises a N501Y substitution.

10. The particle of claim 1, wherein said polypeptide comprises the substitutions:
(i) I358F, N460K, E484K, S494P, Q498R and N501Y;
(ii) I358F, N460K, E484K, Q498R and N501Y;
(iii) I358F, E484K, Q498R and N501Y;
(iv) I358F, V445K, N460K, I468T, T470M, S477N, E484K, Q498R and N501Y; or
(v) I358F, V367W, R408D, K417V, V445K, N460K, I468T, T470M, S477N, E484K, Q498R and N501Y.

11. The particle of claim 1, wherein said polypeptide comprising an amino acid sequence at least 99% identical to SEQ ID NO: 38, 39 or 40.

12. The particle of claim 1, wherein said polypeptide comprises no more than 250 amino acids of the S1 subunit of the spike protein of SARS CoV-2.

13. The particle of claim 1, wherein said polypeptide is a dimer.

14. A method of treating an ACE2-associated disease in a subject in need thereof, comprising administering to the subject a therapeutically effective amount of a particle having an ACE2 targeting moiety attached to an outer surface thereof, wherein said ACE2 targeting moiety comprises a polypeptide comprising an amino acid sequence of SARS CoV-2 receptor-binding domain (RBD), wherein said amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein said polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein said polypeptide comprises at least 170 amino acids of said RBD, wherein said particle is attached to, or encapsulates, a therapeutic agent, thereby treating the disease.

15. A method of diagnosing an ACE2-associated disease in a subject in need thereof, comprising administering to the subject an effective amount of a particle having an ACE2 targeting moiety attached to an outer surface thereof, wherein said ACE2 targeting moiety comprises a polypeptide comprising an amino acid sequence of SARS CoV-2 receptor-binding domain (RBD), wherein said amino acid sequence comprises a modification at position 358 and at least two additional modifications at two positions selected from the group consisting of 484, 498 and 501, wherein the numbering of the positions of the modifications is according to UniProtKB-P0DTC2 (SEQ ID NO: 37), wherein said polypeptide binds soluble, monomeric angiotensin-converting enzyme 2 (ACE2) receptor when expressed on the surface of yeast cells with at least 50 fold higher affinity than the wild-type RBD having an amino acid sequence as set forth in SEQ ID NO: 45, when assayed under identical conditions, wherein said polypeptide comprises at least 170 amino acids of said RBD, wherein said particle is attached to, or encapsulates, a diagnostic agent, thereby diagnosing the disease.

16. The method of claim 14, wherein said disease is a respiratory disease.

17. The method of claim 14, wherein said administering is effected by inhalation.

18. The method of claim 14, wherein said respiratory disease is a respiratory infection.

19. The method of claim 14, wherein said therapeutic agent is selected from the group consisting of a polynucleotide agent, a polypeptide agent and a small molecule agent.

20. The method of claim 14, wherein said therapeutic agent is selected from the group consisting of an antiviral agent, an antibiotic, a cytotoxic agent, a bronchodilator and an anti-inflammatory agent.


SEQUENCE LISTING (48 sequences)

SEQ ID NO: 1 - DNA, 58 bp, synthetic construct
  misc_feature 1..58: note = Single strand DNA oligonucleotide
  source 1..58: mol_type = other DNA
ggcggtagcggaggcggagggtcggctagccattgcccttttggtgaagtttttaacg

SEQ ID NO: 2 - DNA, 76 bp, synthetic construct
  misc_feature 1..76: note = Single strand DNA oligonucleotide
  source 1..76: mol_type = other DNA
ctattacaagtcctcttcagaaataagcttttgttcggatccttcaaaagaaagtactactactctgtatggttgg

SEQ ID NO: 3 - DNA, 55 bp, synthetic construct
  misc_feature 1..55: note = Single strand DNA oligonucleotide
  source 1..55: mol_type = other DNA
ggcggtagcggaggcggagggtcggctagccatccaaacatcaccaacctgtgtc

SEQ ID NO: 4 - DNA, 63 bp, synthetic construct
  misc_feature 1..63: note = Single strand DNA oligonucleotide
  source 1..63: mol_type = other DNA
ctattacaagtcctcttcagaaataagcttttgttcggatccctttggtccacacactgtggc

SEQ ID NO: 5 - DNA, 53 bp, synthetic construct
  misc_feature 1..53: note = Single strand DNA oligonucleotide
  source 1..53: mol_type = other DNA
ggcggtagcggaggcggagggtcggctagccatagggtccaaccaacagagag

SEQ ID NO: 6 - DNA, 67 bp, synthetic construct
  misc_feature 1..67: note = Single strand DNA oligonucleotide
  source 1..67: mol_type = other DNA
ctattacaagtcctcttcagaaataagcttttgttcggatccgaagttcacacacttgttcttcacc

SEQ ID NO: 7 - DNA, 57 bp, synthetic construct
  misc_feature 1..57: note = Single strand DNA oligonucleotide
  source 1..57: mol_type = other DNA
ctgctttggctgctccagctaatggtcatatgtgcccttttggtgaagtttttaacg

SEQ ID NO: 8 - DNA, 61 bp, synthetic construct
  misc_feature 1..61: note = Single strand DNA oligonucleotide
  source 1..61: mol_type = other DNA
gcccgaaccacctccaccagaggatccttcaaaagaaagtactactactctgtatggttgg

SEQ ID NO: 9 - DNA, 45 bp, synthetic construct
  misc_feature 1..45: note = Single strand DNA oligonucleotide
  source 1..45: mol_type = other DNA
tgcccttttggtgaagtttttaacgccaccaggtttgcctctgtc

SEQ ID NO: 10 - DNA, 54 bp, synthetic construct
  misc_feature 1..54: note = Single strand DNA oligonucleotide
  source 1..54: mol_type = other DNA
ttcaaaagaaagtactactactctgtatggttggtagcccactccattggttgg

SEQ ID NO: 11 - DNA, 24 bp, synthetic construct
  misc_feature 1..24: note = Single strand DNA oligonucleotide
  source 1..24: mol_type = other DNA
ctacaaactgcctgatgacttcac

SEQ ID NO: 12 - DNA, 22 bp, synthetic construct
  misc_feature 1..22: note = Single strand DNA oligonucleotide
  source 1..22: mol_type = other DNA
gtccaggttgttgctgttccag

SEQ ID NO: 13 - DNA, 75 bp, synthetic construct
  misc_feature 1..75: note = Single strand DNA oligonucleotide
  source 1..75: mol_type = other DNA
ccatgttcgtgtttctggtgctgctgcctctggtgtccagcaccaacctgtgcccttttggtgaagtttttaacg

SEQ ID NO: 14 - DNA, 87 bp, synthetic construct
  misc_feature 1..87: note = Single strand DNA oligonucleotide
  source 1..87: mol_type = other DNA
ccatgttcgtgtttctggtgctgctgcctctggtgtccagcaccccaaacatcaccaacctgtgcccttttggtgaagtttttaacg

SEQ ID NO: 15 - DNA, 79 bp, synthetic construct
  misc_feature 1..79: note = Single strand DNA oligonucleotide
  source 1..79: mol_type = other DNA
cgacttaagatcgatgcggccgcgagctcgaattttatcaatggtgatggtgatggtgctttggtccacacactgtggc

SEQ ID NO: 16 - DNA, 58 bp, synthetic construct
  misc_feature 1..58: note = Single strand DNA oligonucleotide
  source 1..58: mol_type = other DNA
cgccaccaggtttgcctctgtctatgcctgggaaaggaagaggtttagcaactgtgtg

SEQ ID NO: 17 - DNA, 55 bp, synthetic construct
  misc_feature 1..55: note = Single strand DNA oligonucleotide
  source 1..55: mol_type = other DNA
caggaagaggtttagcaactgtgtggctgactggtctgtgctctacaactctgcc

SEQ ID NO: 18 - DNA, 60 bp, synthetic construct
  misc_feature 1..60: note = Single strand DNA oligonucleotide
  source 1..60: mol_type = other DNA
gaagaggtttagcaactgtgtggctgactactcttggctctacaactctgcctccttcag

SEQ ID NO: 19 - DNA, 65 bp, synthetic construct
  misc_feature 1..65: note = Single strand DNA oligonucleotide
  source 1..65: mol_type = other DNA
cttcagcaccttcaagtgttatggagtgagcccacgtaaactgaatgacctgtgtttcaccaatg

SEQ ID NO: 20 - DNA, 57 bp, synthetic construct
  misc_feature 1..57: note = Single strand DNA oligonucleotide
  source 1..57: mol_type = other DNA
ctgactcctttgtgattaggggagatgaggtggatcagattgcccctggacaaacag

SEQ ID NO: 21 - DNA, 58 bp, synthetic construct
  misc_feature 1..58: note = Single strand DNA oligonucleotide
  source 1..58: mol_type = other DNA
gggagatgaggtgagacagattgcccctggagccacaggcaagattgctgactacaac

SEQ ID NO: 22 - DNA, 60 bp, synthetic construct
  misc_feature 1..60: note = Single strand DNA oligonucleotide
  source 1..60: mol_type = other DNA
ggtgagacagattgcccctggacaaacaggcgttattgctgactacaactacaaactgcc

SEQ ID NO: 23 - DNA, 58 bp, synthetic construct
  misc_feature 1..58: note = Single strand DNA oligonucleotide
  source 1..58: mol_type = other DNA
gacagcaaggtgggaggcaactacaactacctctttagactgttcaggaagagcaagc

SEQ ID NO: 24 - DNA, 57 bp, synthetic construct
  misc_feature 1..57: note = Single strand DNA oligonucleotide
  source 1..57: mol_type = other DNA
ggtgggaggcaactacaactacctctacagacgtttcaggaagagcaagctgaaacc

SEQ ID NO: 25 - DNA, 56 bp, synthetic construct
  misc_feature 1..56: note = Single strand DNA oligonucleotide
  source 1..56: mol_type = other DNA
gagtgaagggcttcaactgttacttcccactcatgtcctatggcttccgaccaacc

SEQ ID NO: 26 - DNA, 58 bp, synthetic construct
  misc_feature 1..58: note = Single strand DNA oligonucleotide
  source 1..58: mol_type = other DNA
ccaggctggcagcacaccatgtaatggagtgcgcggcttcaactgttacttcccactc

SEQ ID NO: 27 - DNA, 56 bp, synthetic construct
  misc_feature 1..56: note = Single strand DNA oligonucleotide
  source 1..56: mol_type = other DNA
ctgttacttcccactccaatcctatggcttccatccaacctatggagtgggctacc

SEQ ID NO: 28 - DNA, 58 bp, synthetic construct
  misc_feature 1..58: note = Single strand DNA oligonucleotide
  source 1..58: mol_type = other DNA
cttcccactccaatcctatggcttccgaccaacctttggagtgggctaccaaccatac

SEQ ID NO: 29 - DNA, 57 bp, synthetic construct
  misc_feature 1..57: note = Single strand DNA oligonucleotide
  source 1..57: mol_type = other DNA
cctatggcttccgaccaacctatggagtgggctggcaaccatacagggtggtggtgc

SEQ ID NO: 30 - DNA, 58 bp, synthetic construct
  misc_feature 1..58: note = Single strand DNA oligonucleotide
  source 1..58: mol_type = other DNA
ccaaccatacagggtggtggtgctgtcctttgaaatgctccatgcccctgccacagtg

SEQ ID NO: 31 - DNA, 58 bp, synthetic construct
  misc_feature 1..58: note = Single strand DNA oligonucleotide
  source 1..58: mol_type = other DNA
gtgaagggcttcaactgttactttccactccaaccttatggcttccgaccaacctatg

SEQ ID NO: 32 - DNA, 66 bp, synthetic construct
  misc_feature 1..66: note = Single strand DNA oligonucleotide
  source 1..66: mol_type = other DNA
gcaagaagggaggcaactacaactacctctacagacgtttcaggaagagcaaactgaaaccatttg

SEQ ID NO: 33 - protein, 139 aa, synthetic construct
  REGION 1..139: note = Protein sequence of modified eUnaG2
MLEKFVGTWKIESSENFGEYLKAIGAPKELADAGDATTPVLYISQKDGDKMTVKIENGPPTFLDTQVSFKLGEEFDEFPSDRRKGVKSVVNLSGEKLVYVQKWDGKETTYVREIKDGKLVVTLTMGDVVAVRSYRRASE

SEQ ID NO: 34 - DNA, 420 bp, synthetic construct
  misc_feature 1..420: note = Nucleic acid sequence of modified eUnaG2
  source 1..420: mol_type = other DNA
atgttagaaaaatttgttggcacctggaagatcgaatcctctgaaaattttggtgaatacttgaaggctatcggtgccccaaaagaattggctgatgctggtgatgctactactccagtcttgtacatttctcaaaaggatggtgataagatgaccgtcaagattgaaaacggtccaccaacttttttggatacccaagtttctttcaagttgggtgaagaattcgacgaatttccatccgatagaagaaagggtgttaagtccgttgttaacttgtctggtgagaagttggtttacgttcaaaagtgggatggtaaagaaaccacttacgtcagagaaatcaaggacggtaaattggttgttactttgaccatgggtgatgttgttgctgttagatcttatagaagggcctctgaataa

SEQ ID NO: 35 - protein, 125 aa, synthetic construct
  REGION 1..125: note = DnbALFA amino acid sequence
MSGEVQLQESGGGLVQPNGSLRLSCNASGVTISALNAMAMGWYRQAPGERRVMVAAVSERGNAMYRESVKGRFTISRDNANNMVSLQMDNLKPEDTAVYYCHVLEDRGDTFHDYWGQGTQVTVSS

SEQ ID NO: 36 - DNA, 375 bp, synthetic construct
  misc_feature 1..375: note = DnbALFA nucleic acid sequence
  source 1..375: mol_type = other DNA
atgagcggtgaagttcagctgcaagaaagcggtggtggtctggttcagcctaatggtagcctgcgtctgagctgtaacgcaagcggtgttaccattagcgcactgaatgcaatggcaatgggttggtatcgtcaggcaccgggtgaacgtcgtgttatggttgcagcagttagcgaacgtggtaatgcaatgtatcgtgaaagcgttaaaggtcgttttaccatttctcgtgataatgctaacaatatggttagcctgcagatggataatctgaaaccggaagataccgcagtgtattattgtcatgttctggaagatcgtggtgatacttttcatgattattggggtcagggcacccaggttaccgttagcagc

SEQ ID NO: 37 - protein, 1273 aa, Severe acute respiratory syndrome-related coronavirus
  source 1..1273: note = Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - protein, 196 aa, synthetic construct
  REGION 1..196: note = B52 amino acid sequence
TNLCPFGEVFNATRFASVYAWNRKRFSNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSKLKPFERDISTEIYQAGSTPCNGVKGFNCYFPLQPYGFRPTYGVGYQPYRVVVLSFELLHGPATVCGPK

SEQ ID NO: 39 - protein, 196 aa, synthetic construct
  REGION 1..196: note = B62 amino acid sequence
TNLCPFGEVFNATRFASVYAWNRKRFSNCVADYSVLYNSASFSTFKCYGVSPTKLNDLCFTNVYADSFVIRGDEVRQIAPGQTGKIADYNYKLPDDFTGCVIAWNSNNLDSKKGGNYNYLYRLFRKSKLKPFERDTSMEIYQAGNTPCNGVKGFNCYFPLQSYGFRPTYGVGYQPYRVVVLSFELLHAPATVCGPK

SEQ ID NO: 40 - protein, 196 aa, synthetic construct
  REGION 1..196: note = B71 amino acid sequence
TNLCPFGEVFNATRFASVYAWNRKRFSNCVADYSWLYNSASFSTFKCYGVSPTKLNDLCFTNVYADSFVIRGDEVDQIAPGQTGVIADYNYKLPDDFTGCVIAWNSNNLDSKKGGNYNYLYRLFRKSKLKPFERDTSMEIYQAGNTPCNGVKGFNCYFPLQSYGFRPTYGVGYQPYRVVVLSFELLHAPATVCGPK

SEQ ID NO: 41 - protein, 66 aa, synthetic construct
  REGION 1..66: note = linker sequence GPGcP amino acid sequence
AGSGGSGGSGGSPVPSTPPTPSPSTPPTPSPSGGSGNSSGSGGSPVPSTPPTPSPSTPPTPSPSAS

SEQ ID NO: 42 - protein, 55 aa, synthetic construct
  REGION 1..55: note = linker NGL amino acid sequence
SGGGGSGGGGNGSNGSGGSNGSNGSGGSNGSNGSGGSNGSNGSGGSGGGGSGGGG

SEQ ID NO: 43 - protein, 455 aa, synthetic construct
  REGION 1..455: note = Dimer amino acid sequence
TNLCPFGEVFNATRFASVYAWNRKRFSNCVADYSVLYNSASFSTFKCYGVSPTKLNDLCFTNVYADSFVIRGDEVRQIAPGQTGKIADYNYKLPDDFTGCVIAWNSNNLDSKKGGNYNYLYRLFRKSKLKPFERDTSMEIYQAGNTPCNGVKGFNCYFPLQSYGFRPTYGVGYQPYRVVVLSFELLHAPATVCGPKAGSGGSGGSGGSPVPSTPPTPSPSTPPTPSPSGGSGNSSGSGGSPVPSTPPTPSPSTPPTPSPSASCPFGEVFNATRFASVYAWNRKRFSNCVADYSWLYNSASFSTFKCYGVSPTKLNDLCFTNVYADSFVIRGDEVDQIAPGQTGVIADYNYKLPDDFTGCVIAWNSNNLDSKKGGNYNYLYRLFRKSKLKPFERDTSMEIYQAGNTPCNGVKGFNCYFPLQSYGFRPTYGVGYQPYRVVVLSFELLHAPATVCGPK

SEQ ID NO: 44 - protein, 60 aa, synthetic construct
  REGION 1..60: note = Ace2 amino acid sequence
MSSSSWLLLSLVAVTAAQSTIEEQAKTFLDKFNHEAEDLFYQSSLASWNYNTNITEENVQ

SEQ ID NO: 45 - protein, 40 aa, synthetic construct
  REGION 1..40: note = Arg319-Phe541 of SEQ ID NO: 37
GIYQTSNFRVQPTESIVRFPNITNLCPFGEVFNATRFASV

SEQ ID NO: 46 - DNA, 588 bp, synthetic construct
  misc_feature 1..588: note = B52 nucleotide sequence
  source 1..588: mol_type = other DNA
accaacctgtgcccttttggtgaagtttttaacgccaccaggtttgcctctgtctatgcctggaacaggaagaggtttagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaaactgaaaccatttgagagggacatcagcacagagatttaccaggctggcagcacaccatgtaatggagtgaagggcttcaactgttactttccactccaaccctatggcttccgaccaacctatggagtgggctaccaaccatacagggtggtggtgctgtcctttgaactgctccatggccctgccacagtgtgtggaccaaag

SEQ ID NO: 47 - DNA, 588 bp, synthetic construct
  misc_feature 1..588: note = B62 nucleotide sequence
  source 1..588: mol_type = other DNA
accaacctgtgcccttttggtgaagtttttaacgccaccaggtttgcctctgtctatgcctggaacaggaagaggtttagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaagaagggaggcaactacaactacctctacagactgttcaggaagagcaaactgaaaccatttgagagggacaccagcatggagatttaccaggctggcaacacgccatgtaatggagtgaagggcttcaactgttactttccactccaatcctatggcttccgaccaacctatggagtgggctaccaaccatacagggtggtggtgctgtcctttgaactgctccatgcccctgccacagtgtgtggaccaaag

SEQ ID NO: 48 - DNA, 579 bp, synthetic construct
  misc_feature 1..579: note = B71 nucleotide sequence
  source 1..579: mol_type = other DNA
tgcccttttggtgaagtttttaacgccaccaggtttgcctctgtctatgcctggaacaggaagaggtttagcaactgtgtggctgactactcttggctctacaactctgcctccttcagcaccttcaagtgttatggagtgagcccaaccaaactgaatgacctgtgtttcaccaatgtctatgctgactcctttgtgattaggggagatgaggtggatcagattgcccctggacaaacaggcgttattgctgactacaactacaaactgcctgatgacttcacaggctgtgtgattgcctggaacagcaacaacctggacagcaagaagggaggcaactacaactacctctacagactgttcaggaagagcaaactgaaaccatttgagagggacaccagcatggagatttaccaggctggcaacacgccatgtaatggagtgaagggcttcaactgttactttccactccaatcctatggcttccgaccaacctatggagtgggctaccaaccatacagggtggtggtgctgtcctttgaactgctccatgcccctgccacagtgtgtggaccaaag